(19) United States
(12) Patent Application Publication
(10) Pub. No.: US 2018/0344950 A1
(43) Pub. Date: Dec. 6, 2018

(54) TRIGGERED QUANTAL DRUG DELIVERY DEVICE, METHOD AND SYSTEM ("TQD3")

(71) Applicant: ALEXANDER PHILIP GOUMENIOUK, GAINESVILLE, FL (US)

(72) Inventors: ALEXANDER PHILIP GOUMENIOUK, BOWEN ISLAND (CA); PETER ALEXANDER GOUMENIOUK, BOWEN ISLAND (CA); Benjamin Walter WILD, RICHMOND (CA)

(21) Appl. No.: 15/778,943

(22) PCT Filed: Nov. 25, 2016
(86) PCT No.: PCT/CA2016/051394

§ 371 Date: May 24, 2018

Related U.S. Application Data
(60) Provisional application No. 62/260,279, filed on Nov. 26, 2015.

(51) Int. Cl. A61M 11/00

(52) U.S. Cl. CPC A61M 11/007; A61M 2205/609; A61M 2205/8225; A61M 2210/0618; A61M 2210/1067; A61M 2205/0222

(57) ABSTRACT

This invention is designed and intended to provide simple, precise delivery of a variety of medications to mucosal surfaces of a living subject, human or non-human animal, including but not limited to the nasal or rectal mucosa, which sites afford rapid absorption and therefore onset of effect of the thus delivered medication. Such medications include, but are not limited to, anticonvulsants, drugs to treat anaphylactic shock, antidotes for poisoning, and medications which are required urgently where either a parenteral formulation is not available or an i.v. route is difficult. Simplicity and needle free safety of use of the various embodiments of the invention allows its operation by laypersons, school-teachers, nurses, paramedics and non¬medical persons in e.g. the armed service. Modular embodiments of the invention facilitate mixing and matching of delivery systems with different dosages and types of medications.

### 1.0 FIELD OF THE INVENTION

Drug delivery device, system and method for precise delivery of a defined quantity of drug to various human or non-human animal mucosal surfaces when triggered.

### 2.0 BACKGROUND OF THE INVENTION

The device, system and method according to this invention is useful for delivering a wide variety of drugs to mucosal surfaces of a patient, (human or non-human animal), in need of such treatment, primarily via nasal or rectal routes of delivery, particularly, but not exclusively, when other more conventional routes of medication delivery (intravenous, intraperitoneal, subcutaneous, etc.) are unavailable, compromised or where mucosal delivery presents distinct advantages over other routes of administration.

In particular, in emergency situations, where time is of the essence, and/or when access to conventional routes of drug administration (e.g. intravenous or “i.v.” access) is already hampered, or where a patient presents with difficult to access vasculature, e.g. in obese patients, paediatric patients, or patients presenting with hypovolemia or other conditions in which blood vessels lack patency, or in e.g. HIV-infected subjects where accessing the vasculature carries risks, the high vascularity and rapid absorption of drugs via the nasal or rectal mucosa provides a valuable alternative in both humans and animals.

For such situations, a mucosal delivery device, system and method according to this invention is provided as a valuable alternative, according to which a precise amount of a selected drug is delivered via a device which, in one embodiment, is a single-use, user-friendly device which either laypersons or medical personnel could benefit by having on hand in case of need.

Situations in which implementation of an embodiment of this invention is indicated include, but are not limited to, (i.e. naming but a few):
Intranasal insulin delivery for a host of indications over and above and/or independent of systemic regulation of glycaemic state—see, for example, “Nasal Insulin”, (Downloaded, on 21 Nov. 2015 and to be filed in an IDS, from http://www.alzforum.org/therapeutics/nasal-insulin).
Newly approved naloxone intranasal delivery applications—see “FDA moves quickly to approve easy-to-use nasal spray to treat opioid overdose”, Nov. 18, 2015, (Downloaded, on 21 Nov. 2015 and to be filed in an IDS, from http://www.fda.gov/NewsEvents/Newsroom/PressAnnouncements/ucm473505.htm
Delivery of Compounds via the “Nose Brain Pathway”, whereby medication appropriately deployed intra-nasally contacts the olfactory mucosa, resulting in molecular transport directly across this tissue and into the cerebral spinal fluid (see, “Therapeutic Intranasal Drug Delivery: Needleless treatment options for medical problems”, downloaded on 21 Nov. 2015 and to be filed in an IDA, from http://intranasal.net/overview/default.htm

Accordingly, embodiments of this invention as described herein below are directly poised to provide benefits via intra-nasal or intra-rectal delivery of medications or delivery to other mucosal surfaces.

It is believed that the present invention provides a novel and inventive device, method, kit and system for delivery of precise dosages of medication to select mucosal surfaces via triggered release of a gas driven piston deploying a precise dose of medication to said surface(s).

### 3.0 SUMMARY OF THE INVENTION

A needle free device is provided whereby a selected dose of a medication is delivered to the mucosa of a recipient (human or animal) by a gas-cylinder driven piston. In various embodiments, the device is adapted, or adaptable, for delivery to a particular mucosal surface, including, but not limited to, e.g. the nasal mucosa or the rectal mucosa. In various embodiments, the system is modular, permitting the user to select a particular gas canister, a particular trigger or activation mechanism, a particular medication and dosage, and a conduit adapted for optimal delivery of the dosage of the desired medication to either the nasal vestibule or the rectum. In other embodiments, the device comprises a pre-selected trigger/activation mechanism, medication, dose and conduit. Permutations and combinations of the various embodiments disclosed herein will occur to those skilled in the art based on the present disclosure, and such permutations and combinations are to be considered as coming within the scope of this invention.

The device, system and method according to this invention takes advantage of mucosal routes of administration of medicines that are currently underutilized. Specifically, the nasal and rectal mucosa are highly vascularized and therefore afford a rapid route of administration of drugs that are either not available in intravenous preparations or in situations where intravenous administration is difficult.

Current “nasal sprays” expose only a small fraction of the nasal mucosa to aqueous drug. The present system exposes a much larger area of nasal mucosa to medication, affording a more rapid and complete delivery of a precision dose of drug. The device is an operator friendly system that requires either a quarter turn of the exterior cylinder for flipping open a protective cap and depressing a single button to trigger delivery of medication. Either of these procedures results in the complete discharge of a precise amount of drug. A unique venturi system at the tip of the device provides a nozzle for nebulization of the aqueous drug into particles between 20 μm and 200 μm in diameter, depending on the included venturi ducts. The terms “nebulize”, “nebulization”, “atomize”, “atomization” including when used in reference to a nozzle or the droplets that are formed, are used interchangeably herein. These droplet sizes are deemed optimal for nasal mucosa absorption. The entire system is also user-friendly insofar as there is a “skirt” on the nozzle, similar to interchangeable earbuds for in-ear headphones, that allows for a complete seal to be made in nostrils varying from pediatric size to large adult nostrils.

Thus, the nasal delivery device, allows for the administration of urgently needed medications by persons that range from laypersons to ICU nurses or paramedics without requiring intravenous access. The pharmacokinetics of this route of administration is comparable to intravenous delivery exceeds the efficiency and ease of administration using known inhalational delivery devices.

Considerable flexibility is provided by different embodiments according to this invention, according to which application nozzles adapted for different routes of mucosal administration are interchangeable, as are the doses and composition of medications.

### 4.0 BRIEF DESCRIPTION OF THE DRAWINGS

FIG. 1 is a photographic representation of the limited delivery of medication to the nasal vestibule achieved by prior art medication delivery devices.

FIG. 2 is a detailed, side-sectional view and cross-section top view of the components of a first embodiment, 100 of the TQD3 device according to this invention adapted for nasal administration of nebulized liquid medication comprising a pushbutton mechanism for triggering nasal delivery of medication.

FIG. 3 is a side-sectional view of embodiment 100 showing the relevant steps in preparation and use of that device.

FIG. 4 is a side-sectional view and a top section view of the components of a second embodiment, 200 of the TQD3 device according to this invention adapted for nasal administration of nebulized liquid medication comprising a quarter-turn screw mechanism for triggering nasal delivery of medication.

FIG. 5 is a side-sectional view of embodiment 200 showing the relevant steps in preparation and use of that device.

FIG. 6 is a side-sectional view showing the components of a third embodiment 300 of the TQD3 device according to this invention comprising a two-component modular system for assemblage of the unitary and operative device for nasal delivery of medication.

FIG. 7 is a side-section view showing components of a fourth embodiment 400 of the TQD3 device according to this invention comprising a two-component modular system for assemblage of the unitary and operative device for nasal delivery of medication.

FIG. 8 is a side-section view showing components of a fifth embodiment 500 of the TQD3 device according to this invention comprising a two-component modular system for assemblage of the unitary and operative device for nasal delivery of medication.

FIG. 9 is a side-section view showing components of a sixth embodiment 600 of the TQD3 device according to this invention comprising a two-component modular system for assemblage of the unitary and operative device for rectal delivery of medication.

FIG. 10 is a side-section and top transverse section view showing components and providing guidance dimensions of the sixth embodiment 600 of the TQD3 device according to this invention comprising a two-component modular system for assemblage of the unitary and operative device for rectal delivery of medication.

FIG. 11 is a side-section view of embodiment 600 showing the relevant steps in preparation and use of that device.

FIG. 12 is a side-section view showing components of a seventh embodiment 700 of the TQD3 device according to this invention comprising a two-component modular system for assemblage of the unitary and operative device for rectal delivery of medication.

FIG. 13 is a side-section and top transverse sectional view of embodiment 700 providing guideline dimensions for components of the device.

FIG. 14 is a side-sectional view of embodiment 700 showing the steps in assemblage of components to form the unitary and operative device for rectal delivery of medication.

FIG. 15 is a side-sectional view of an eighth embodiment 800 of the TQD3 device according to this invention comprising a two-component modular system for assemblage of the unitary and operative device for rectal delivery of medication.

FIG. 16 is a side-sectional view of an ninth embodiment 900 of the TQD3 device according to this invention comprising a two-component modular system for assemblage of the unitary and operative device for rectal delivery of medication.

FIG. 17 is a side-sectional view of an tenth embodiment 1000 of the TQD3 device according to this invention comprising a two-component modular system for assemblage of the unitary and operative device for rectal delivery of medication.

### 5.0 DETAILED DISCLOSURE OF THE PREFERRED EMBODIMENTS OF THE INVENTION

### 5.1 Introduction

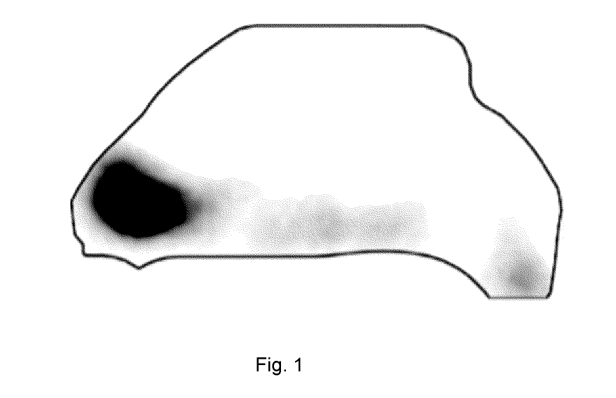

With respect to embodiments of this invention for nasal delivery of medication, unlike conventional “nasal sprays”, the mucosal delivery device according to this invention provides particle sizes which may be varied according to the optimal particle size of the medication in question and selection of appropriate components of the device (e.g. high or low pressure gas canisters and design of the conduit through which medication is delivered) from 20 μm to 200 μm in diameter at sufficient pressure and velocity to cover a considerably larger proportion of the nasal mucosa (between about 2 to 20 fold). Current so-called “metered dose delivery systems” actually expose only a small fraction of the nasal mucosa to the drug in question due to lack of pressure and/or velocity. The actual dose from these devices is imprecise both in droplet size and volume delivered, each affecting the actual dose administered. FIG. 1 herein shows the limited degree of medication delivery using a known nasal delivery device. By contrast, the device of this invention dispenses a precise and predictable amount of drug to a large area of the nasal mucosa bilaterally, even though inserted into only one nostril. As further described herein below, a unique and flexible membrane sheath, similar to variable sized earbuds on headphones, allows for a precise seal into that particular nostril. The pressure utilized for nasal drug delivery is safe but sufficient to distribute the drug throughout the oropharynx and nasal membranes bilaterally. We have found a pressure in the range of 1-40 PSI, which those skilled in the art will be able to optimize for a given application and route of delivery. A pressure of 1-10 PSI is considered sufficient for rectal delivery of medications using the device, method and system of this invention, while higher pressures, up to about a 40PSI maximum, is considered desirable for delivery of medications to the nasal mucosa in a nebulized/aerosolised form. Of course, these are pressures in the canister, not at the point of delivery and those skilled in the art may find optimal pressures within and even beyond these limits which are appropriate for a given medication or route of delivery. The high vascularity of the nasal mucosa allows for rapid absorption of appropriately sized particles and molecules such that the pharmacokinetics are comparable to pulmonary inhalation of substances such as, for example, nicotine. It has been known for some time that the nasal mucosa is a rich bed of vascularity for administration of bioactive compounds of various descriptions. For example, see Sutherland et al., “Nasal nicotine spray: a rapid nicotine delivery system”, Psychopharmacology, 1992:108(4):512-8. This demonstrates the potential for the rapid and emergent treatment of conditions without the need for intravenous (i.v.) delivery, particularly in situations where i.v. administration is hindered, such, e.g. drugs where intravenous preparations are not available or an intravenous route is not accessible. Conditions for use of the present device include, but are not limited to, seizures, anaphylactic reactions and nerve gas poisoning. There is evidence in the literature (see, for example, Hanson and Frey, “Intranasal delivery bypasses the blood-brain barrier to target therapeutic agents to the central nervous system and treat neurodegenerative disease”, BMC Neurosciece 2008, 9(Suppl 3); S5) that delivery of certain medications to the nasal mucosa enhances delivery to the brain. Accordingly, in some embodiments, because of a nasal route of delivery, medication is directly administered to the brain, maximizing blood-brain barrier passage. This device does not require a needle or access to intravenous or intra-arterial routes of administration, making the device, in one embodiment, disposal, and without production of biohazard. Thus the ease of administration, precision of dosing, pharmacokinetic profile and route of administration distinguish it from current art. In a device according to the present invention, a child of elementary school age could administer a life-saving therapeutic agent with accuracy, precision and effectiveness due to the ease of administration, as further described in detail herein below. A further advantage of the device and system according to this invention is that certain embodiments afford single hand use to dispense medication to appropriate mucosal surfaces, including in the nose or rectum.

With respect to embodiments of this invention for rectal administration of medication, also interchangeably referred to as “per rectum”, the invention takes advantage of the high vascularity of the rectal mucosa to administer medication via a rectal route. The device according to this embodiment of the invention comprises a pneumatic discharge unit which drives a hydraulic plunger to deliver medicine stored in a medication storage cylinder. The amount of medication contained in the medication storage cylinder is varied depending on the cavity size and the length of the plunger. In one embodiment, also true for intranasal delivery, color-coding is utilized, e.g. green for the lowest dose, yellow for a moderate dose and red for the highest dose. The exterior of the medicine storage cylinder is labeled to indicate both the quantity of the medication and its name. In one embodiment, the medicine storage cylinder is attached to said pneumatic discharge device by means of a screw mount, a clip or the equivalent. In a preferred embodiment, the medicine storage cylinder is covered by a removable protective sheath to maintain sterility. In a preferred embodiment, between the protective sheet and the medicine storage cylinder there is provided sterile lubricant jelly. The end of the medicine storage cylinder is covered by a frangible membrane designed to rupture on actuation/triggering of the pneumatic discharge. Once the sheath is removed, the device is inserted through the anal canal. Its length (about 80 mm, but those skilled in the art will know that this length may be varied based on experience and need without departing from the scope of this invention) is sufficient to have the tip protrude into the rectal space allowing the delivery of the liquid via the pneumatic plunger device into the rectum where it is absorbed.

As described herein below in further detail, whether for nasal or rectal delivery, in one embodiment, a pneumatic discharge device is triggered by removal of a protective cover and depression of a button to drive a compressed gas cylinder to rupture, thereby releasing pressure to drive a pneumatic plunger. In another embodiment, a quarter screw-turn in a first direction, e.g. clockwise, drives the compressed gas cylinder to rupture, and a counter-clockwise turn releases the compressed gas pressure to drive the pneumatic plunger. The compressed gas is then vented to the atmosphere when the desired medication dose has been delivered.

Table 1 herein below provides a summary guide as to the various embodiments of this invention as further described in detail herein below:

TABLE 1
Permutations and combinations of the TQD3 Device:

| TQD3 | | | |
|---|---|---|---|
| EMBODI- | | | |
| MENT | TRIGGER | GAS CYLINDER | CONDUIT |
| 100 | Protected | High pressure -> 20 micron | Nasal |
| | Pushbutton | to 200 micron droplets | |
| 200 | Screw Turn | High pressure -> 20 micron | Nasal |
| | | to 200 micron droplets | |
| 300 | Protected | High pressure -> 20 micron | Nasal |
| | Pushbutton | to 200 micron droplets | |
| 400 | Screw Turn | High pressure -> 20 micron | Nasal |
| | | to 200 micron droplets | |
| 500 | Screw Turn | High pressure -> 20 micron | Nasal |
| | | to 200 micron droplets | |
| 600 | Protected | Low pressure for rectal | Rectal |
| | Pushbutton | delivery | |
| 700 | Screw Turn | Low pressure for rectal | Rectal |
| | | delivery | |
| 800 | Screw Turn | Low pressure for rectal | Rectal |
| | | delivery | |
| 900 | Screw Turn | Low pressure for rectal | Rectal |
| | | delivery | |
| 1000 | Protected | Low pressure for rectal | Rectal |
| | Pushbutton | delivery | |

### 5.2 Nasal Medication Delivery Embodiments of the Invention

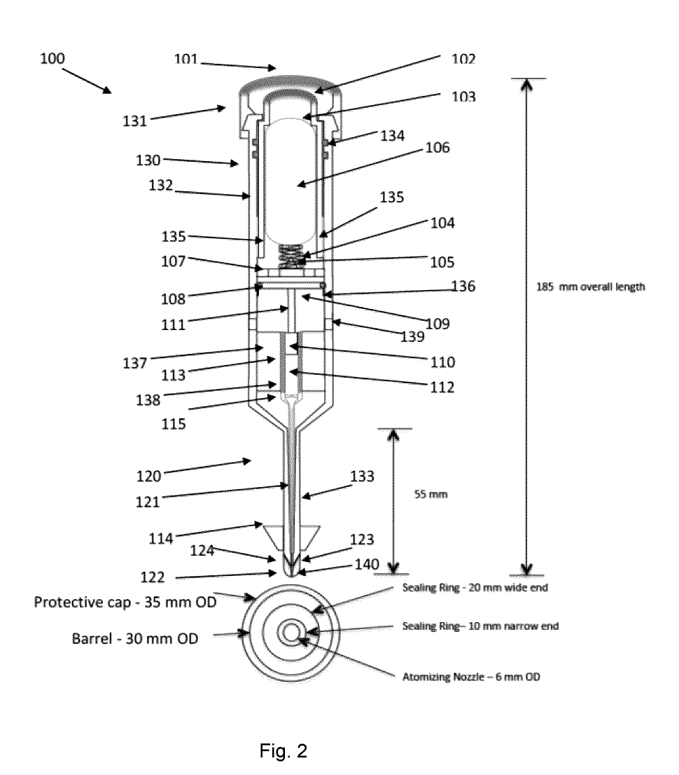

Referring now to FIG. 2, a first embodiment 100 of the TQD3 device is described in some considerable detail. Other embodiments are then described by difference, in relation to and with reference to this embodiment, with like elements retaining the numbering provided for elements in this embodiment throughout, but without necessarily labelling and numbering every element labeled and numbered in this representation. In certain representations, only such common indicia for common or like elements as are needed to provide orientation to the reader are included, along with additional elements which differ as between embodiments.

Said first embodiment 100 of the TQD3 device comprises a pushbutton activator/trigger 102 disposed beneath and protected by a removable protective cover 101, which is adapted to be flipped off with the thumb of one hand or otherwise removed to expose the pushbutton activator/trigger 102. When the pushbutton activator 102 is depressed, a compressed gas cylinder 103 is forced against a spring 104 onto a lance or rupture pin 105. The spring 104 biases the gas cylinder 103 away from the lance 105 to thereby permit release of pressurized gas 106 from the gas cylinder 103. The expelled pressurized gas 106 then travels through a perforated support plate 107, which, in a preferred embodiment, also supports the lance 105, such that the pressurized gas 106 discharged from the gas cylinder 103 drives pneumatic actuator 109 sealed at its edges abutting the inside of housing 130 by O-ring 108, to drive hydraulic piston 110 connected to said pneumatic actuator 109 via shaft 111 to advance piston 110 thereby dispensing a precise amount of medication 112 stored in a substantially cylindrical medication storage depot 113 through progressively narrowing conduit 120. The distal end of the conduit 120 tube is, in one embodiment, narrow enough for insertion into pediatric nostrils to accommodate the distal end of tube 120. To ensure a good seal to prevent loss of medication dosage from the nose, including for pediatric or adult nostrils, a deformable soft foam or rubber skirt 114 is provided at the distal end of the conduit 120 to ensure an air-tight seal prior to activation of the trigger 102 to initiate delivery of the medication 112. The skirt 114 is, in a preferred embodiment, removable and replaceable with any of a series of skirts of greater or lesser diameter, selected as appropriate to the needs of a given patient's nostril dimensions. Skirts of various dimensions may be included in a kit with other components of various embodiments of the device according to this invention (particularly with respect to modular embodiments described herein below), to enable maximum flexibility for the user and different subjects.

Prior to triggering of the actuation mechanism (perforation of the gas cylinder as described above), the medication is retained in the medication storage depot 113, which is preferably a sealed tube, cylinder, capsule, or like medication containment structure, made from any appropriate material suitable to maintain the integrity of the medication. The walls of the medication storage depot 113 may be composed of plastic, polypropylene, polyethylene, gelatin, or an other material known in the art suitable for this purpose and depending on the nature of the medication to be stored therein, and its desired shelf-life. A first end of the medication storage depot 113 is sealed by the distal end of the hydraulic piston 110, and at the other by a friable rupture disc 115 which breaks when the compressed gas 106 is released by the trigger mechanism 102 to drive the hydraulic piston 110 forward into the medication storage depot 113. The diameter of the medication storage depot 113 is adapted to accommodate different dosages of medication 112 as is the diameter of hydraulic piston 110, allowing the piston travel distance to remain constant as between different dosages required to be delivered.

The progressively narrowing conduit 120 has several functions defined by its structure. First, it accommodates a passageway/connecting tube 121 for the medication 112 to travel through to the interior of the nostril cavity once the rupture disk 115 is ruptured. Second, at the distal end 122 of the of the conduit 120 there is provided an atomizing nozzle 123 which, depending on the viscosity of the medication 112 and the amount of pressure transmitted from the gas cylinder 103 when ruptured, produces droplets from between about 20 microns to about 200 microns in diameter, with every intermediate diameter being facilitated by variations in gas pressure, viscosity of the medication, and geometry of the elements of the atomizing nozzle 123, including the at least one venturi 124. The desired droplet diameter is under the control and selection of the user by selection of a TQD3 device with a compressed gas cylinder 103 matched or match-able to a particular medication formulation to produce optimally absorbable droplets of medication with diameters anywhere between and including 20-200 microns. Routine experimentation by those skilled in the art will permit appropriate matching of a given medication formulation and optimal drop sizes and gas canister pressures to achieve desired medication delivery and absorption parameters. In a preferred embodiment, droplets appropriate for nasal delivery, are produced by at least one and preferably at least two rear-facing venturi duct(s) 124 included in the distal end 122 of the conduit 120 that cause air to aerosolize or nebulize the liquid into the appropriate particulate diameters. The geometry of the venture ducts 124 impacts on the geometry of the droplet particles produced by the device, between and including the 20 μm to 200 μm diameter range.

The above-described elements of the TQD3 device 100 are contained within housing 130 comprising a top or proximal end 131, a substantially cylindrical body, 132, manufactured from plastic, metal, or other suitable material, and a distal end 133, comprising a tapered housing element which contains and supports the progressively narrowing conduit 120. The degree of tapering of the conduit is engineered for each specific drug to adjust for fluid velocity as it passes the venturi ducts to generate the correct particulate size of nebulization for the drug in question. The long axis of housing 130 runs from the proximal end 131 to the distal end 133 thereof. The housing 130 may be unitary or modular, and in various embodiments, certain contained elements differ in the housing structure to accommodate different modes of operation and assembly of the TQD3 device of this invention. In general, however, the housing 130 is of a shape and dimension to accommodate a pressurized gas canister 106, and double O-ring pressure seals 134 within the housing at the proximal end, between the inside of the housing the exterior of the gas canister 103 to prevent escape of gas when released from the canister 103 except to ensure that the gas drives the pneumatic actuator 109 and connected piston 110 to effect delivery of the medication 112. In the embodiment 100, in addition, there are provided positioning guides 135 (preferably at least three such guides are provided, even though only two are shown in the two-dimensional representation of FIG. 2) to ensure the motion of the gas cylinder 103 is smooth and aligned with the long axis of the the device 100. Preferably, the guides 135 are contoured around the top (proximal end) of the gas cylinder 103 as shown in the figure so as to retain the gas cylinder 103 in place with the assistance of biasing spring 104, while, at the same time, providing a sliding containment for the gas cylinder 103 when the pushbutton trigger 102 is either depressed or released.

To prevent premature movement of the pneumatic actuator 109 and piston 110 into the medication storage depot 113, there is provided at least one pneumatic actuator restraining catch 136 and preferably several such pneumatic actuator restraining catches 136, or said actuator restraining catch 136 may be formed as a continuous ring. On triggering the TQD3 device by depression of said pushbutton actuator 102 and release of pressurized gas from said pressurized gas canister 106, the O-ring seal 108 becomes deformed enough to permit the pneumatic actuator 109 to pass beyond said actuator catch 136 to initiate expulsion of the medication 112 from said medication storage depot 113.

Within the internal structure of the housing 130, defining the side wall structure of the medication storage depot 113, there is provided a cylinder 137 concentric with the housing body 130 with a bore 138 defined therein to accommodate said piston 110 and said medication storage depot 113 below said piston 110. Said cylinder 137 and bore 138 provide the function not only of containing said medication in a precise dose 112 within said cylindrical medication storage depot 113, but also of guiding said piston 110 through said medication storage depot 113 to efficiently eject the medication 112 therefrom. Said cylinder 137 also provides the function of a stop to prevent further travel of the pneumatic actuator 109 beyond what is necessary to fully dispense the dose of medication 112 from the medication storage depot 113 and out of the conduit 120. Concurrent with the pneumatic actuator 109 reaching the end of its travel, by coming into contact with the top of said cylinder 137, built up gas pressure trapped behind the pneumatic actuator 109 is vented from the housing via at least one and preferably more than one depressurizing vent(s) 139. The depressurizing vents 139 allow gas to vent from the housing, thereby releasing built-up pressure and ensuring safety on disposal of the device.

Housing 130 tapers to a point at the distal end of said housing to support application of the sealing skirt 114 on the outer distal surface thereof, to protect said conduit 120, to include the at least one, and preferably at least two, venturi port(s) 124, and to provide an orifice 140 from which medication droplets exit into the inside of a patient's nostril.

As described herein above, the embodiment 100 of the TQD3 device of the invention is, in one embodiment, a single use medical delivery device designed to deliver precise amounts of medication to the nasal mucosa. The delivery device is adaptable for a range of medications and application requirements, including fluid viscosity, total volume delivered and droplet size, by varying the compressed air cylinder pressure and the diameter of the hydraulic piston. The delivery device is preferably supplied in a sterile protective wrap or container, complete and ready to use, including prescription specific medication and several sizes of soft foam nostril sealing skirts to accommodate variability in patient nostril sizes, optionally in the form of a kit.

The delivery device is designed to deliver between (and including) about 0.5 ml to about 1.5 ml of medication, atomized to a droplet size ranging from about 20 microns to about 200 microns. Naturally, those skilled in the art will appreciate that other volumes may be accommodated without departing from the invention as herein disclosed and claimed. The driving force needed to atomize the medication is provided by release of the compressed air from the gas cylinder and is applied to the pneumatic actuator driving the hydraulic piston that forces the medication, first through a rupture disc, then to a narrowing connecting tube, and finally to an atomizing nozzle discharging prescription specific medication onto the nasal mucosa membranes of the patient.

Preferred dimensions of the TQD3 device embodiment 100 are also shown in FIG. 2, with a cross-sectional view shown below the device showing various preferred diameters of the device elements depicted above the cross section and as described herein above. It will be understood by those skilled in the art that the dimensions provided are by way of guidance rather than by way of limitation, and those skilled in the art will appreciate that embodiments of this invention incorporating modifications in these dimensions do not depart from the scope of the appended claims, provided such modifications continue to incorporate the operative principles disclosed herein.

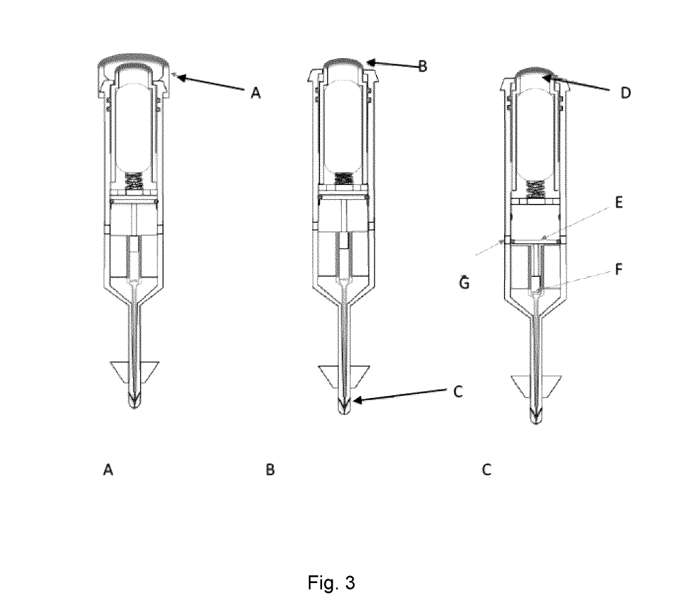

With reference to the above description of device elements, the following narrative with reference to additional figures is provided in which like numerals are utilized to show relevant parts to those shown in FIG. 2. The narrative minimizes use of reference numerals, to preserve readability. To prepare the device 100 for use, the user removes it from any protective wrap or container in which it is provided, selects a soft foam nostril-sealing ring or skirt as appropriate to a given patient's nostril dimensions, positions the skirt on the discharge conduit to suit the patient and then removes the protective cap 101. The conduit is then positioned in the nostril of the patient. Once it is confirmed that the soft foam ring forms a seal with the nostril to ensure that atomized droplets remain in the nasal cavity of the patient, the user then triggers release of medication by depressing the pushbutton activator/trigger 102, driving the compressed air cylinder into the puncture pin/lance 105 creating an opening for the compressed gas to discharge, see FIG. 3, A-C, in which steps A-G reflect the order of steps taken to utilize the device according to the method of this invention. Thus, in step A, the protective cap is removed. In step B, the atomizing nozzle is positioned inside the patient's nostril. In step C, the trigger pushbutton is activated to initiate puncture of the gas canister In step D, the pushbutton is released to thereby permit compressed gas to escape from the ruptured gas cylinder. In step E, once sufficient pressure from released gas has built up to impel the pneumatic actuator to move beyond the restraining catch, the pneumatic actuator drives the piston through the medication storage depot, rupturing the rupture disc, and causing medication to discharge. In step F, the rupture disc is ruptured in such a way that none of the rupture disc material breaks off to potentially clog the atomizing nozzle. In step G, the compressed gas is permitted to vent to the atmosphere, allowing for safe disposal of the de-pressurized device. Those skilled in the art will appreciate that the order of steps disclosed is not limiting on the invention. Thus, in certain instances, the order of, e.g. steps B and C may be reversed, provided that the trigger pushbutton is not released prior to insertion of the device into patient's nostril.

When the user releases the push button activator, the spring 104 biases the gas canister away from the lance 105 to permit compressed gas to escape. The user holds the nasal sprayer in position until all medication has discharged. The perforations in the support plate allow passage of the compressed air to drive the pneumatic actuator forward to move the piston. The double O-ring pressure seal prevents leakage of compressed air from the housing.

To ensure all the medication is nebulized to the required droplet size, the pneumatic actuator remains fixed in position by the pneumatic actuator restraining catch until the pressure has increased sufficiently to ensure proper functioning of the atomizing nozzle. Once the pneumatic actuator pressure is high enough, the pneumatic actuator O-ring deforms and passes over the pneumatic actuator restraining catch, causing the piston to increase the pressure in the medication storage depot, causing the rupture disc to fail and drive the medication through the connecting tube to the atomizing nozzle. The connecting tube narrows towards the atomizing nozzle, significantly increasing the velocity of the medication. High velocity fluid passes through the atomizing nozzle mixing with air drawn through the venturi ducts and forming droplets of the specified size.

The pneumatic actuator travels until it reaches the depressurizing vents and strikes the plastic support positioning the medication storage depot. The compressed air driving the pneumatic actuator follows the path of least resistance and discharges from the delivery device housing, thereby fully depressurizing the entire devise. At this point the prescribed volume of medication has been delivered into the patient's sinus cavity and the delivery device can now be removed from the patient and safely discarded. The volume of medication in the storage cylinder is calculated to include the volume of medication prescribed plus the volume left in the connecting tube and atomizing nozzle once the hydraulic piston travel has stopped. Adjusting the diameter of the medication storage cylinder (and piston) varies the volume of medication delivered. The pressure requirement in the compressed air cylinder varies according to atomized droplet size and the volume of medication to be delivered.

To protect the medication from exposure and to ensure the shelf life of the delivery device, the medication storage cylinder is sealed at one end by the rupture disc and at the other end by a seal laminated to the bottom of the hydraulic piston. The rupture disc is scored or otherwise weakened in the middle, at the sides or at any other aspect thereof, to ensure predictable failure of the rupture disc once the hydraulic piston starts moving, without, at the same time, releasing any debris which might clog the conduit through which the medication must travel to reach the inside of the nose.

Depending on the atomized droplet size required and the working pressures needed to create the droplet size, the pneumatic actuator, hydraulic piston, medication storage cylinder, connecting tube and the discharge nozzle can be made from either plastic or stainless steel. Producing a 20-micron droplet requires higher pressure than producing a 200-micron droplet. The compressed air cylinder and the positioning spring are preferably made of steel or other resilient and corrosion resistant material. The other pieces of the TQD3 device are preferably manufactured from plastic via injection molding, 3D printing, or any other appropriate process known to those skilled in the art.

The atomizing nozzle is preferably designed to produce a full cone spray pattern to limit the transfer of energy to the nasal mucosa while permitting deep penetration of the atomized droplets into the sinus cavity. The maximum diameter of the discharge conduit and atomizing nozzle is 6 mm, allowing use of the device on a wide range of patients.

Once prepared for operation, the TQD3 device is designed to work using one hand freeing the other hand to accurately locate the atomizing nozzle. At the higher working pressures required for smaller atomized droplets, in the event a user finds the level of effort required to puncture the cylinder with push button activation too challenging, an alternate trigger mechanism, described next, which implements a quarter turn screw cap, rather than a pushbutton trigger, is provided. Such an embodiment requires two-hand operation.

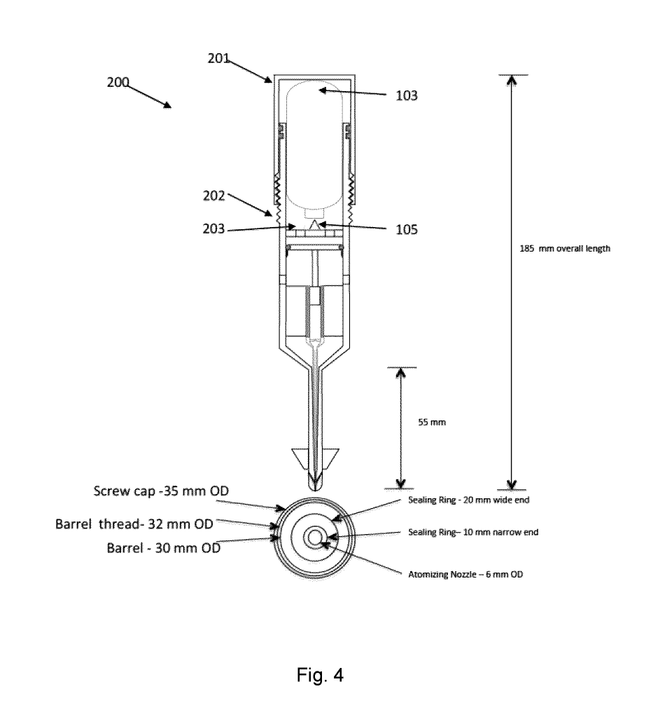

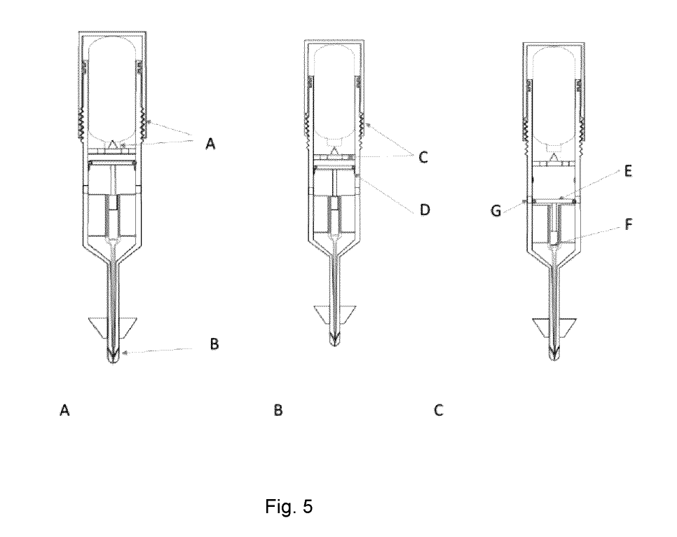

Referring now to FIG. 4, there is shown a second embodiment 200 of the TQD3 device in which like numbered elements are identical to those described herein above for embodiment 100, but reference numerals are kept to a minimum to maintain a clear representation of this embodiment. Instead of a pushbutton trigger, in embodiment 200, there is provided a quarter turn rotational screw trigger mechanism, and, in this embodiment, there is no need for biasing spring 104 included in embodiment 100. In this embodiment, the protective cap 101 is replaced by a top housing element 201 comprising threads which mate with threads 202 on the exterior proximal end of the housing to thereby complete the structure of the housing while at the same time providing an alternate trigger mechanism to that included in embodiment 100. The proximal end of the gas cylinder 103 abuts the interior underside of housing element 201 such that, depending on the direction of the mating screw threads, an approximately quarter turn of said housing element 201 in relation to threads 202 results in the gas cylinder 103 being driven into lance 105. In this embodiment, the distal end of gas cylinder 103 preferably includes a sealing ring 203 to prevent premature discharge of compressed gas while the user positions the conduit end of the device into a nostril of the patient. Once correctly positioned and the sealing skirt has been confirmed to make a good seal with the nostril, the user turns, approximately a quarter turn, said housing element 201 back toward its original position, to thereby back the lance 105 out of the puncture made in the gas cylinder 103 to release gas from the cylinder. All other aspects of embodiment 200 and its mode of use and operation are identical to those described herein above in relation to embodiment 100. Similar to that shown in relation to embodiment 100, in this figure, cross section and longitudinal dimensions are provided below and alongside the main representation of embodiment 200. In FIG. 5A-C, there is shown the sequence of steps relevant to use of this embodiment of the invention: In step A, the threaded cap is rotated to drive the compressed air cylinder into the puncturing pin 105. The sealing ring 203 prevents premature discharge of compressed air while the user positions the atomizing nozzle inside the patient's nostril, as indicated at B. In Step C, once the atomizing nozzle is positioned in the patient's nostril, the threaded cap is rotated back toward it's original position to release compressed air from the now punctured gas canister. Compressed air passes through the perforated support plate to pressurize the pneumatic actuator to drive the piston into and through the medication storage depot. In Step D, the pneumatic actuator restraining catch is still shown preventing advancement of the pneumatic actuator until pressure sufficient to atomize medication to the desired droplet size has been reached. In step E, once the pressure is sufficient, the pneumatic actuator O-ring deforms sufficiently to slide over the pneumatic actuator restraining catch, driving the hydraulic piston through the medication storage cylinder, discharging the medication. In Step F, the rupture disc, designed to fail predictably without any of its constituent material being released to prevent plugging of the atomizing nozzle, is shown having ruptured and released the medication. In Step G, the pneumatic actuator is shown at the end of its travel, and venting of the built-up pressure is vented via the at least one vent, permitting depressurization of the device and, if a single-use embodiment, safe disposal thereof, and if a multi-use embodiment, safe preparation (cleaning, sterilization, recharging medication and recharging and/or replacement of the gas canister, and repackaging) of the device for its next use. In certain embodiments, the gas canister is equipped with a valve mechanism whereby the gas canister may be recharged for further use, without the need to replace the gas canister.

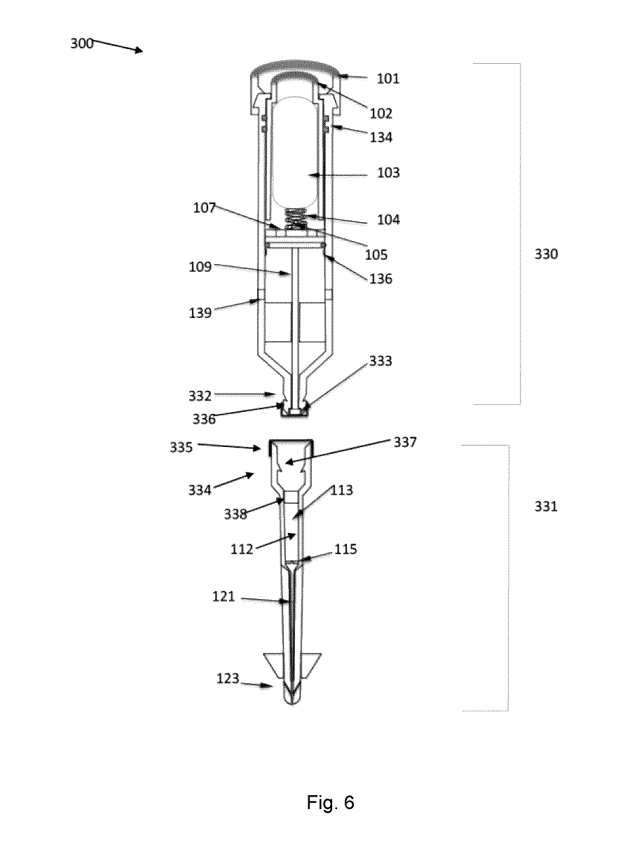

Referring now to FIG. 6, a third embodiment 300 of the TQD3 device of the invention is shown as a modular device in which all elements are identical to embodiment 100 but in the present embodiment, the housing and conduit elements are provided as non-unitary, modular elements which are assembled via a snap on, screw on, or equivalent assembly mechanism. This embodiment provides certain advantages and flexibilities above and beyond those provided by the unitary device. Different upper housing elements, including different gas canisters (e.g. high or low pressure) may be assembled with different conduit elements containing different medications stored in the medication storage depot (e.g. high, medium and low dosages of the same medication or different medications (or mixtures thereof)) according to this embodiment of the invention. As shown in FIG. 6, embodiment 300 includes an upper modular canister unit 330 and a lower modular conduit unit 331. Upper modular canister unit 330 housing terminates at the distal end thereof in a male snap attachment element 332, preferably protected by a protective cover 333 to maintain integrity and cleanliness of the device and internal working parts thereof, prior to attachment to the modular conduit unit 331. Modular conduit unit 331 comprises, at its upper end, a mating female snap attachment element 334, which likewise, is preferably protected by a protective cover 335. The male snap attachment element 332, in a preferred embodiment, includes a circumferential groove 336 into which circumferential internal ridge 337 of the modular conduit unit 331 fits when said upper modular canister unit 330 and said lower modular conduit unit 331 are engaged with each other, after removal of respective protective covers 333 and 335. In this embodiment, the medication storage depot 113 is defined at its upper aspect by piston 338 which is driven to discharge the medication 112 by rupturing the rupture disk 115 via contact between the distal end of the pneumatic actuator 109 and the proximal upper surface of said piston 338. According to this embodiment of the invention, prior to use, the user selects an appropriate upper modular canister unit 330, lower modular conduit unit 331, removes protective covers 333 and 335, snaps the upper canister unit 330 together with the lower conduit unit 331 to thereby form a unitary TQD3 device 300. All other operations thereafter are identical to those described above for embodiment 100.

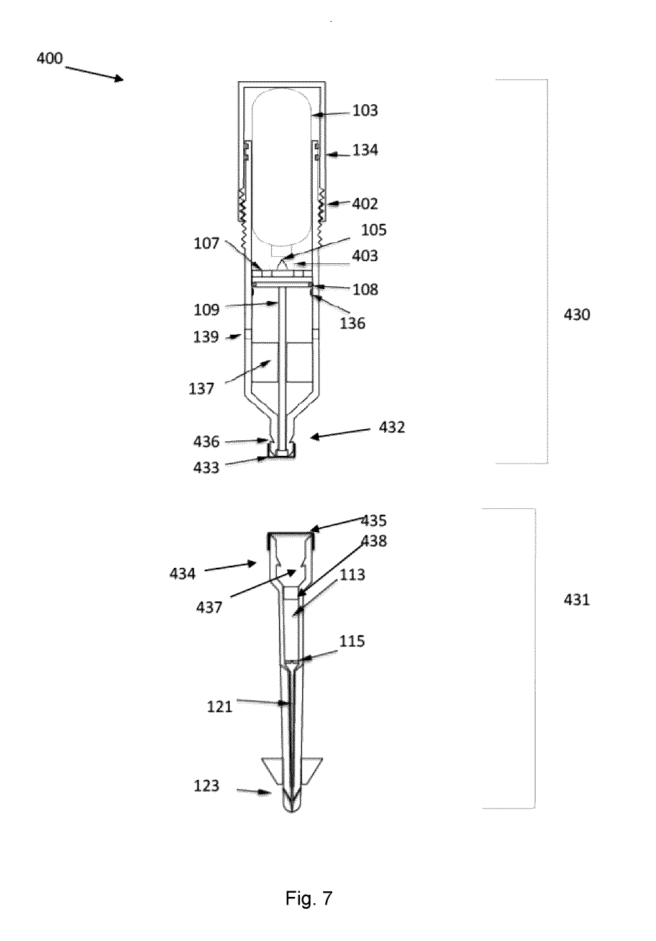

Referring now to FIG. 7, a fourth embodiment 400 of the TQD3 device of the invention is shown as a modular device in which all elements are identical to those for embodiment 300, except that, in the present embodiment, the trigger mechanism and mode of use once assembled is that shown and described above for the quarter turn embodiment 200. Accordingly, as shown in FIG. 7, embodiment 400 comprises an upper modular canister unit 430 and a lower modular conduit unit 431. Upper modular canister unit 430 housing terminates at the distal end thereof in a male snap attachment element 432, preferably protected by a protective cover 433 to maintain integrity and cleanliness of the device and internal working parts thereof, prior to attachment to the modular conduit unit 431. Modular conduit unit 431 comprises, at its upper end, a mating female snap attachment element 434, which likewise, is preferably protected by a protective cover 435. The male snap attachment element 432, in a preferred embodiment, includes a circumferential groove 436 into which circumferential internal ridge 437 of the modular conduit unit 431 fits when said upper modular canister unit 430 and said lower modular conduit unit 431 are engaged with each other, after removal of respective protective covers 433 and 435. In this embodiment, the medication storage depot 113 is defined at its upper aspect by piston 438 which is driven to discharge the medication 112 by rupturing the rupture disk 115 via contact between the distal end of the pneumatic actuator 109 and the proximal upper surface of said piston 438 when the embodiment is assembled by connecting units 430 and 431. According to this embodiment of the invention, prior to use, the user selects an appropriate upper modular canister unit 430, lower modular conduit unit 431, removes protective covers 433 and 435, snaps the upper canister unit 430 together with the lower conduit unit 431 to thereby form a unitary TQD3 device 400. All other operations thereafter are identical to those described above for quarter turn trigger mechanism embodiment 200, utilizing housing body 402 with threads which mate for quarter turn triggering and driving of the gas canister 103 into the lance 105. Sealing ring 403 is included to prevent premature discharge of gas once the gas canister is pierced to release its contents.

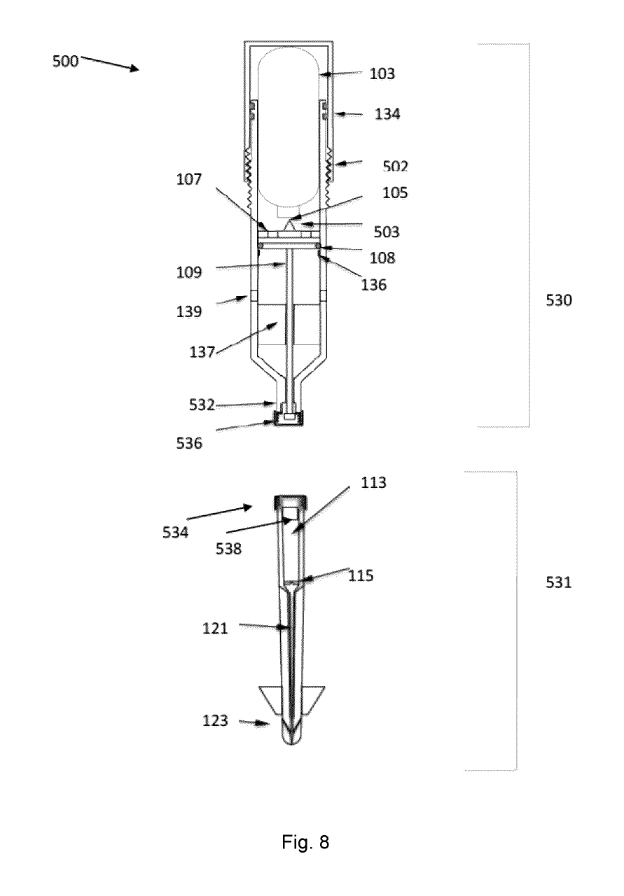

Referring now to FIG. 8, a fifth embodiment, 500 of the TQD3 device of the invention is shown as a modular device in which all elements are identical to those for embodiment 400, except that, in the present embodiment, rather than snap assembly mechanism as shown in FIG. 7 for embodiment 400, in this embodiment, the upper canister unit 530 and lower conduit unit 531 are assembled by a screw-assembly mechanism whereby, at the distal end 532 of upper canister unit 530, a female inner thread 536 is provided which mates with the threaded male upper aspect 534 of the proximal end 534 lower conduit unit 531. Those skilled in the art will appreciate that the screw thread assembly element may be included with a pushbutton trigger embodiment, such as embodiment 400 shown in FIG. 7. Likewise, those skilled in the art will further appreciate that other equivalent assemblage mechanisms, such as a Luer-lock mechanism may be utilized to bring the two modular elements together to form a unitary TQD3 device according to this invention. Likewise, those skilled in the art will appreciate that the element 536 could be fashioned as a male threaded element and element 534 could be fashioned as a female threaded element. As described above, for this embodiment, upper threaded housing element 502 is rotated to engage the distal end of the gas canister 103 with the lance 105. Sealing ring 503 prevents premature release of compressed gas. As with the above-described elements, once sufficient pressure has built up, actuator 109 drives piston 538 into and through the medication reservoir to eject the medication 113 through nozzle 123 once properly emplaced in the nose of a subject.

### 5.3 Rectal Medication Delivery Embodiments of the Invention

The embodiments of the invention described next are designed to take advantage of the rectal route of administration. This has been a neglected route of administration, aside from, e.g., suppositories, which have a slower rate of absorption due to the wax vs liquid delivery media. The rectal mucosal membranes represent a highly vascularized area and, as such, provide a rapid route for medication absorption. This embodiment of the invention is intended to be user-friendly insofar as the user need only select a medicine storage cylinder clearly marked in terms of type of medication and, preferably, color-coded for the dose of medicine in question. The cylinders containing medication preferably contain a variety of doses, preferably clearly marked on the exterior and, again identified by the e.g. green (low), yellow (medium), and red (highest) dose designations. The user assembles the device from its component parts, as further described herein below (and, by analogy, as described above with respect to the modular device for nasal delivery), e.g. by screwing the cylinder onto the delivery device, by clipping it on, or otherwise affixing the parts to each other (e.g. via Luer lock), and then pulls off any protective sheath in which the component parts are provided, leaving a nozzle designed to deliver the medicine through the anal canal into the rectum. The exterior of the nozzle is preferably pre-lubricated with a sterile jelly, allowing for easy insertion without delay, and, therefore, rapid administration of the drug in question. Once triggered, by a quarter turn of an exterior housing on the delivery device, or by pushing a pushbutton (after flipping open a protective cover), the device releases compressed gas driving a piston into the medicine storage cylinder causing the release of liquid or gel from that cylinder into the rectal cavity.

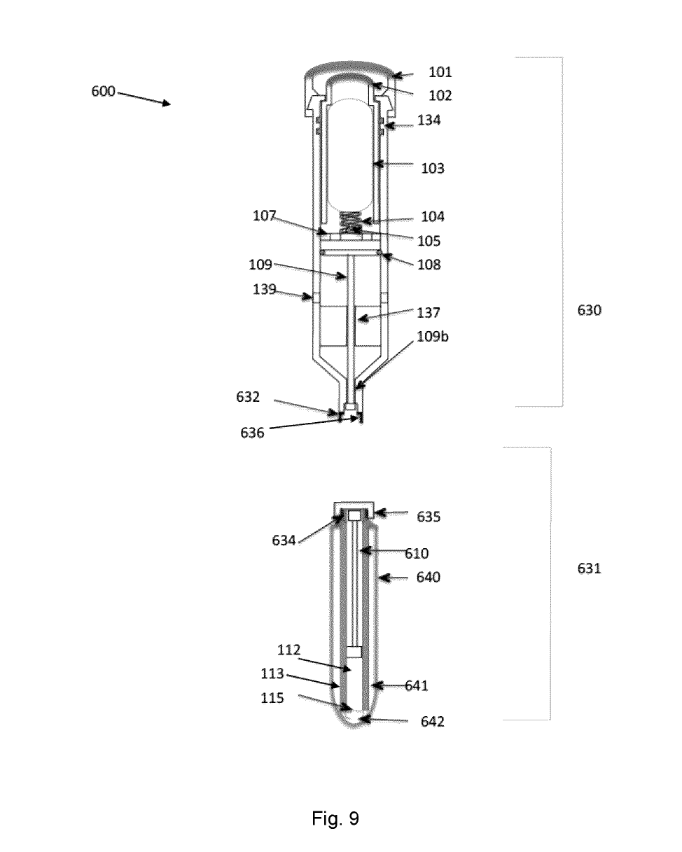

Referring now to FIG. 9, there is provided a sixth embodiment 600 of the invention in which the elements of the device are substantially similar to those shown herein above, e.g. embodiments 300, 400 and 500, with elements in common with embodiment 100 numbered identically. A pushbutton trigger mechanism, a screw-turn trigger mechanism, or the like is included as for the nasal delivery embodiments described above. Like elements are numbered accordingly, without necessarily referring to each described and numbered element by number, as the reader will already be familiar with these elements and numbering from the above-described figures, embodiments and elements.

Differences between nasal and rectal administration of medication according to this invention include that there is no need for the sealing skirt element 114 used in the nasal delivery embodiments, as delivery to the rectum carries much reduced risk of loss of the small volume of medication delivered and the anal sphincter prevents loss of medication once applied. Furthermore, because delivery of medication to the rectum and efficient uptake of the medication is less dependent on the need for producing small droplets of medication, this embodiment of the device, system and method includes a gas cylinder which is of a lower pressure, needed only to deliver the medication without the need to atomize it, and there is no need for the venturi/nebulizer system included in the nozzle of the nasal delivery embodiments described above. Naturally, those skilled in the art will appreciate that such elements may be used in the rectal delivery embodiments without departing from the scope of the invention disclosed and claimed herein, as all reasonable permutations and combinations of the various embodiments disclosed or suggested herein and their equivalents are considered as coming within the scope of the appended claims.

This embodiment of the invention takes advantage of the high vascularity of the rectal mucosa to allow administration of a drug by rectal delivery. With reference to FIG. 9 as well as the operative principals described above with respect to the nasal delivery embodiments of the invention, rectal delivery embodiments of the invention comprise two principal components, the first proximal component of which is a pneumatic discharge unit which drives a hydraulic plunger into the second, distal component comprising a medication storage cylinder, containing the desired medication. The amount of medication contained in the medicine storage cylinder is varied depending on the cavity size and the length of the plunger. In a preferred embodiment, as described above in connection with the nasal delivery embodiments of the invention, the modular elements of this embodiment are color-coded, e.g. with green for the lowest dose, yellow for a moderate dose and red for the highest dose. The exterior of the medicine storage cylinder is labeled to indicate both the quantity of the medication and its name. The medicine storage cylinder is attached to the pneumatic discharge device by means of a screw mount, a clip mount, a Luer lock mechanism or equivalent means known to those skilled in the art. As with the nasal delivery embodiments of the invention such as embodiment 100.

In embodiment 600, there is provided an upper canister unit 630 and lower conduit unit 631 which are assembled by a screw-assembly mechanism whereby, at the distal end 632 of upper canister unit 630, a female inner thread 636 is provided which mates with the threaded male upper aspect 634 of the proximal end of lower conduit unit 631. Those skilled in the art will appreciate that the male and female elements may be reversed, with the female element at the proximal end of conduit unit 631 and the male threaded element at the distal end of upper canister 630. Likewise, those skilled in the art will further appreciate that other equivalent assemblage mechanisms, such as a Luer-lock mechanism or a snap-together mechanism may be utilized to bring the two modular elements together to form a unitary TQD3 device according to this invention. The upper component 630 of this embodiment is substantially identical to the upper portion of described above for embodiment 100, except that in this embodiment, the shaft of the pneumatic actuator 139 is extended as shown at 109b to permit transmission of the gas pressure to the piston 610 included in lower conduit element 631 when the upper 630 and lower 631 components are assembled by screwing the threaded mating elements 636 and 634 to each other and the gas canister 103 is punctured by lance 105. The lower conduit element 631 is preferably protected at its proximal end by removable protective cover 635, which is removed prior to assemblage of element 630 with 631. For ease of insertion into the rectum, the lower conduit unit 631 is preferably protected by a removable protective covering which surrounds a preferably sterile lubricious gel 641 (any of a wide variety of gels are available and known for this purpose, including, but not limited to, e.g. K-Y® Jelly) which remains as a coating after cover 640 has been removed. As with the other embodiments described above, below hydraulic piston 610 and partially defined by the distal aspect thereof, there is medication 112 stored in cylindrical medication storage depot 113 the distal aspect of which, as with the above described embodiments, is retained by rupture disc 115. Finally, rather than the nozzle 123 included in nasal delivery embodiment 100, for rectal administration, a simpler applicator discharge nozzle 642 is adequate, as formation of atomized droplets is not critical for rectal administration of medication.

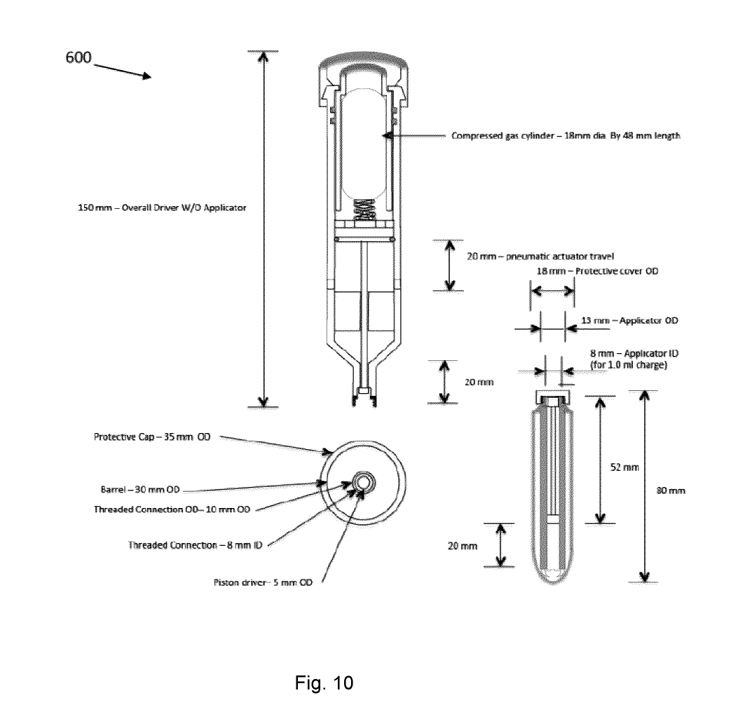

By way of ensuring adequate written description and adequate enablement for those skilled in the art wishing to practice this invention, FIG. 10 provides illustrative rather than limiting dimensions for embodiment 600.

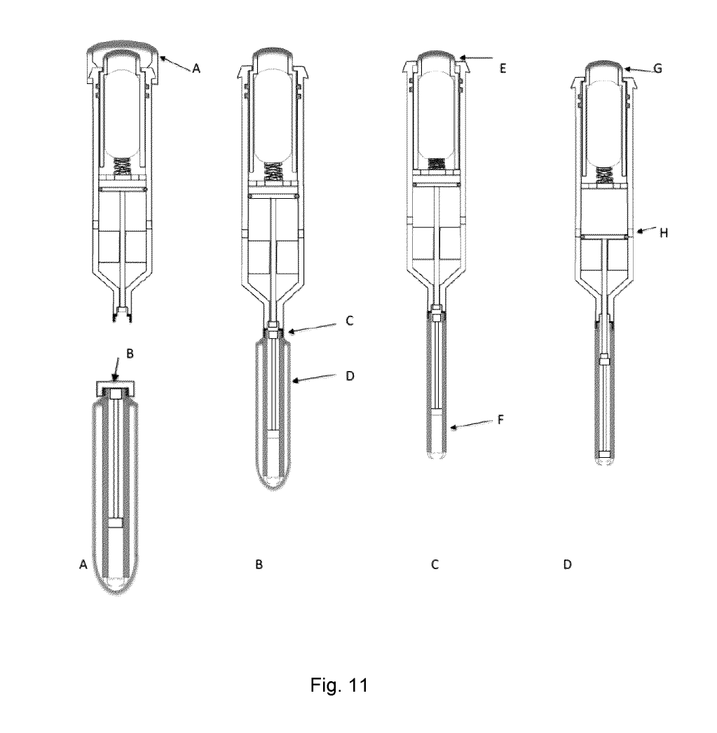

With reference to FIG. 11A-D, the assembly of embodiment 600 is illustrated. In Step A, the pushbutton protective cover 101 is removed and in Step B the protective cover 635 is removed. In Step C, the applicator 631 is screwed together with the upper canister 630 to form the unitary and operational device, and in Step D, the gel filled protective cover is removed from the applicator 631. In Step E, the applicator is inserted into the rectum of the patient and in Step F, the pushbutton trigger is depressed. Of course, once again, provided care is taken in not releasing the pushbutton trigger prior to insertion of the delivery conduit into the rectum, the order of these steps may be reversed. In Step G the pushbutton trigger is released which allows the spring to bias the perforated gas canister away from the lance, thereby allowing pressurized gas to drive the pneumatic actuator and piston to drive the medication into the rectum of the subject. In Step H, once the pressurized gas has been released via the vents, the applicator is removed from the patient and, in a single use embodiment, the spent and depressurized device is safely discarded.

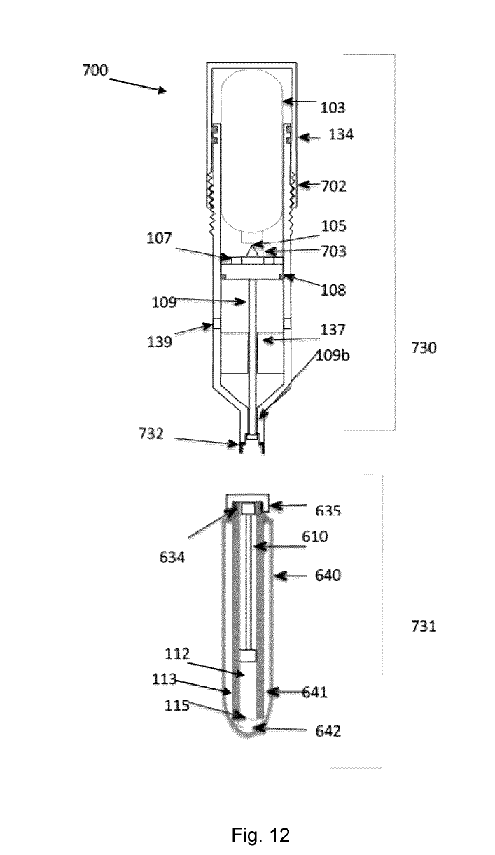

Referring now to FIG. 12, there is provided a seventh embodiment 700 of the invention in which the elements of the device are substantially similar to those shown herein above, e.g. embodiments 300, 400 and 500, with elements in common with embodiment 100 numbered identically. Embodiment 700 incorporates a screw-turn trigger mechanism, as opposed to the push-button trigger included in embodiment 600, and is similar in operation, bearing in mind the differences in description provided herein above between the different embodiments incorporating these different trigger mechanisms. The lower conduit component 731 of this embodiment is substantially identical to the lower conduit component 631 of embodiment 600. Accordingly, the same numbering is included here to avoid the need to repeat the above description here.

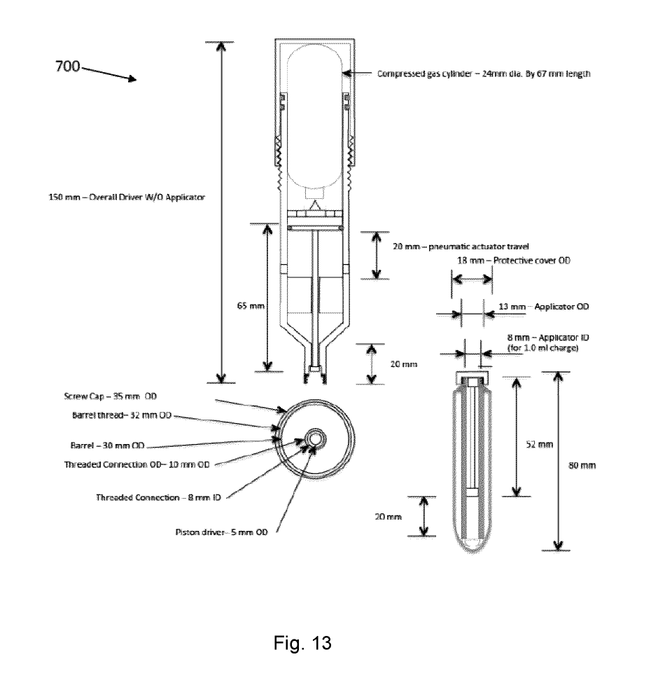

By way of ensuring adequate written description and adequate enablement for those skilled in the art wishing to practice this invention, FIG. 13 provides illustrative rather than limiting dimensions for embodiment 700.

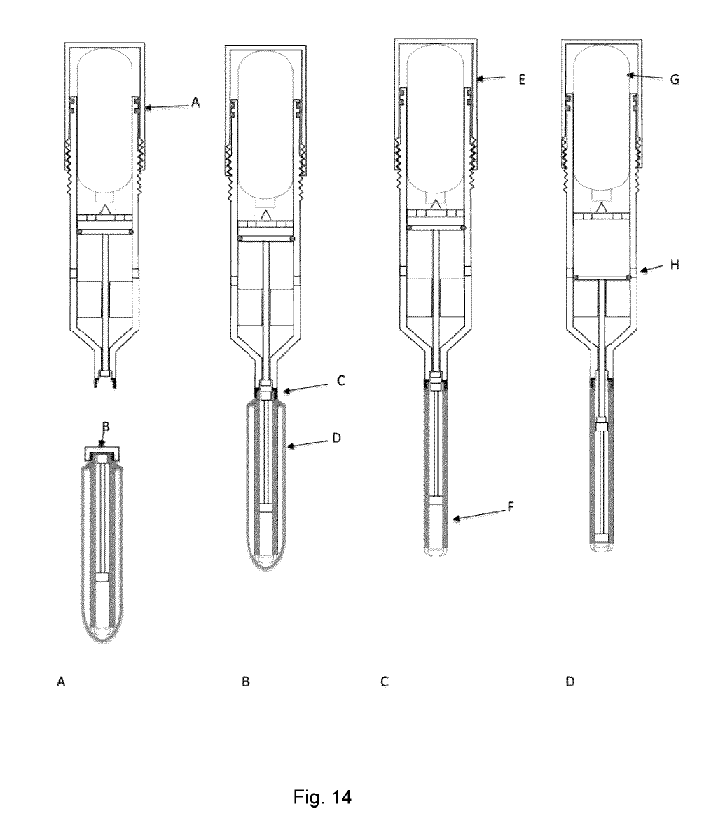

FIG. 14A-D provides a representation of the assembly and use sequence for embodiment 700, which is substantially similar to that illustrated above in FIG. 10 for embodiment 600, bearing in mind, of course, the differences in trigger mechanism included in the two embodiments. In Step A, the TQD3 device is removed from any protective wrap, and in Step B, the protective seal is removed from the applicator. In Step C, the two components 730 and 731 are affixed to each other by means of the screw threads on the mating male and female interlocking parts. In Step D, the protective cover is removed, leaving the gel coating for easy insertion into a patient's rectum. In step E, the applicator is inserted in the patient's rectum, and in Step F, the screw cap is rotated a quarter turn to bring the gas canister into impingement on the lance to rupture the gas canister. In Step G, the screw cap is rotated back toward its original position about a quarter turn, to thereby allow the compressed gas to escape from the perforated canister, and at step H, once the pneumatic actuator has impinged on the plunger, all medication has been delivered to the rectum of the subject and the gas pressure has been vented, the device is removed from the rectum of the subject, and, in a single use embodiment, the device is discarded, and an in a multi-use embodiment, the device is disassembled for cleaning and replacement or recharging with gas. The device is then readied to receive a new, sterile, medication storage canister to be attached or for the existing storage depot to be recharged. Those skilled in the art will appreciate that while shown as modular components for assembly prior to use, this embodiment, and indeed any modular embodiment according to the invention may be provided as a unitary device ready for use. Likewise, any embodiment shown as a unitary device ready for use may be provided as modular components for assembly prior to use, including in the form of a kit.

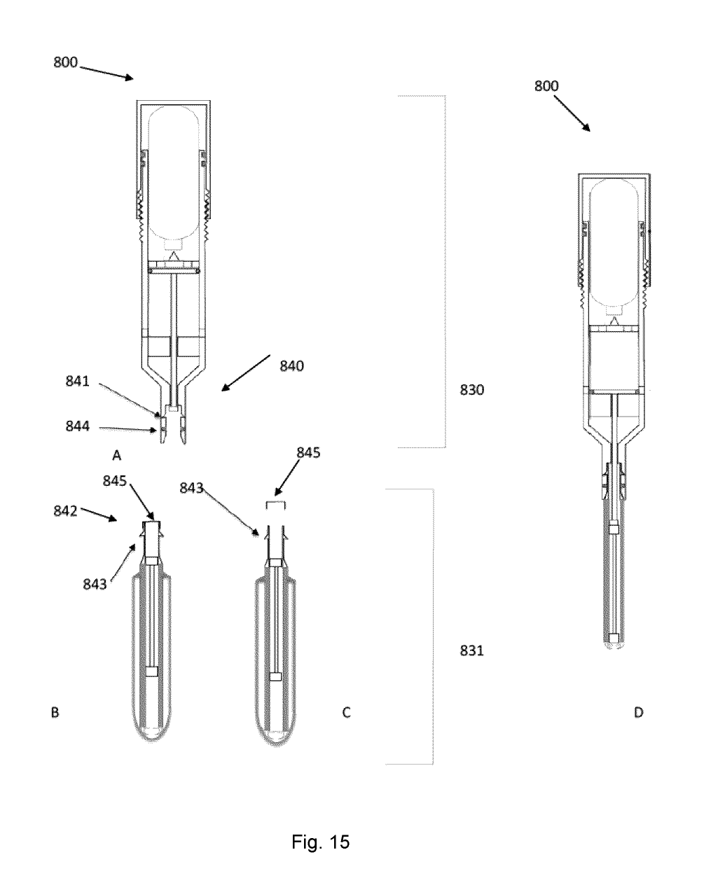

In FIG. 15, an eighth embodiment, 800, of the TQD3 device according to the invention is shown. This embodiment is substantially similar to embodiment 700, except that the assembly mechanism for affixing the upper canister 830 to lower applicator 831 is achieved by latching hook snap-together arrangement, rather than the mating male and female threaded mechanism included in embodiment 700. As all elements of embodiments 800 are substantially identical to those of embodiment 700, FIG. 15 and this description only describe the different fixation mechanism. As can be seen, the distal portion of upper canister 830 terminates in a female receiver 840 comprising a continuous internal notch or groove 841 within the inside surface of the female receiver 840, while lower applicator portion 831 comprises a male component 842 for insertion into said female receiver 840 to which it is affixed by at least one, two, three, or four latching hooks, 843 or a continuous circumferential protrusion around the proximal portion of said male component 842 such that upon insertion into said female receiver 840, said hooks or protrusion 843 snaps into said notch or groove 841, thereby fixing components 830 and 831 to each other. To ensure a gas and fluid-tight seal on snapping components 830 and 831 together, in a preferred embodiment, distal to the groove or notch 841 within female receiver 840, there is provided a receiver O-ring 844. A protective cap or seal 845 is shown protecting the proximal aspect of applicator 831, (FIG. 15B), and once removed (FIG. 15C) in readiness for assembly of parts 830 and 831. FIG. 15D shows embodiment 800 after it has been assembled, triggered for release of medication into the rectum, and after all pressurized gas has been vented from the system in readiness for disposal or recharging.

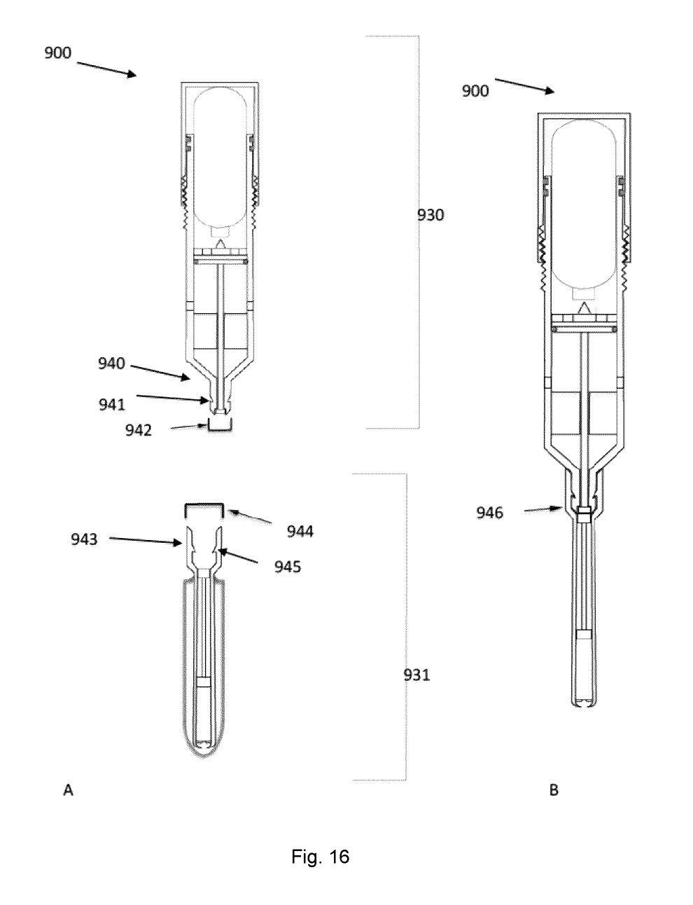

In FIG. 16, a ninth embodiment, 900 of the TQD3 device is shown which is substantially similar to embodiment 800 described above except for a variation on the assembly mechanism for assembling the top canister portion 930 to the lower applicator portion 931. As can be seen, (see FIG. 16A), in this embodiment, canister component 930 terminates at its distal end in an male attachment element 940 comprising a groove 941 and a protector 942 and bottom canister discharge component 931 terminates, at its proximal end, in a female receiver 943 and a protector 944. Internal to said female receiver 943, there is provided a series of hooks 945 for engagement with groove 941 when the protectors 942 and 944 are removed and components 930 and 931 are brought into engagement with each other by inserting male component 940 into female receiver 943. In FIG. 16B, the assembled TQD3 embodiment 900 is shown assembled, with the male/female mechanism in place 946 and the device ready for use.

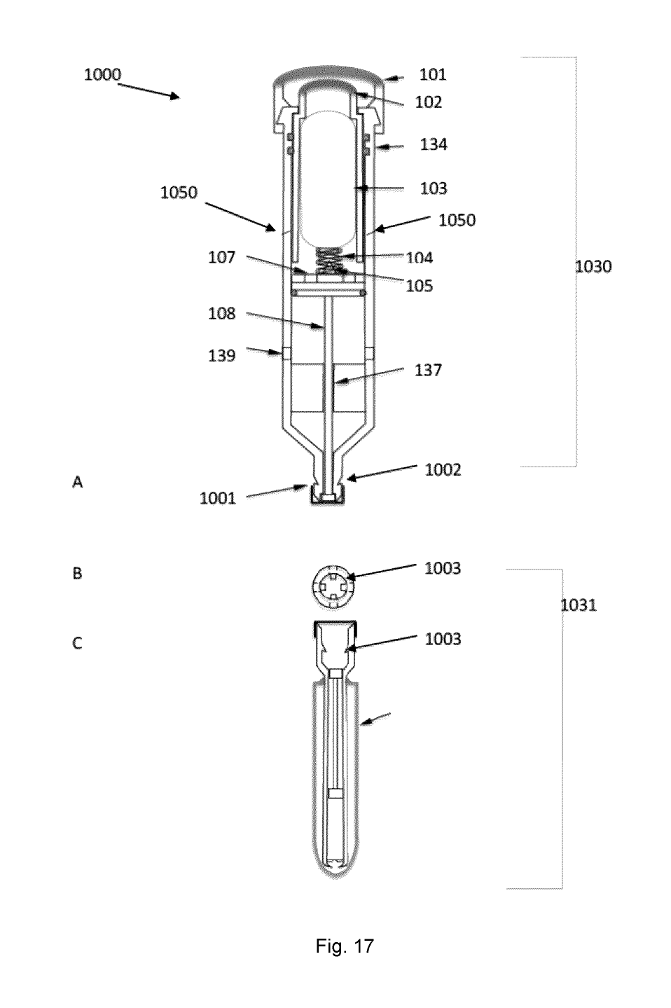

In FIG. 17, a tenth embodiment, 1000, is shown which is substantially similar to embodiment 900, except that in this embodiment, a pushbutton trigger mechanism is utilized. The upper canister portion 1030 terminates in a male connector 1001 comprising a groove 1002 into which snap projections 1003 engage, shown in cross-section in FIG. 17B and in side sectional view in FIG. 17C, when components 1030 and 1031 are snapped together to form a unitary TQD3 device ready for use. Where access to internal components is desired, feature 1050 comprising a detachment mechanism permits access to the internal components of the device, including for replacement or recharging of the gas cylinder.

In each of the foregoing embodiments for rectal delivery of medication in which assembly of components is provided for, preferably, the medication storage element is covered by a protective sheath to retain cleanliness and sterility, if considered necessary, and which is removed prior to use. Preferably, a sterile lubricant gel is included between the protective sheet and the medication applicator. The end of the medicine storage cylinder is preferably covered by a fragile membrane designed to rupture when the pneumatic discharge occurs but not when the protective sheet is removed.

Once the sheath is removed, the device is inserted through the anal canal. Its length (about 80 mm) is sufficient to have the tip protrude into the rectal space, without penetrating too deeply, thereby allowing delivery of the stored medication in liquid or gel form, via triggering of the pneumatic plunger, into the rectum where it is absorbed.

As with the nasal delivery embodiments of the invention, the pneumatic discharge device is triggered either by flipping off a protective cover and depressing a button to cause the rupture of the cylinder containing the gas driving the pneumatic plunger or, alternatively, a quarter screw turn clockwise and then counter clockwise to cause the same release of gas. The gas would then travel through the perforated support plate to drive the piston into the medication storage depot.

### 5.4 General Considerations

The medication delivery system described in this patent disclosure comprises a variety of general-purpose pressurized canisters and applicators that can be combined to deliver a range of medications via patient nasal passages or rectally, also interchangeably referred to herein as “per rectum”. The pressurized gas canister has several variations including High and low pressure compressed air cylinders to suit a variety of delivery requirements such as fine droplets suitable for nasal passage delivery or simple discharge of a liquid or cream per rectum.

Quarter turn screw cap or push button canister activation to accommodate the physical capabilities (e.g. grip strength) of a wide range of health care workers and other users.

Applicator assembly via snap-on, screw-on, or equivalent connector means for low-pressure and high-pressure delivery requirements. Interchangeable nozzles means there is flexibility with respect to the site of delivery, such as nasal or rectal, and the ability to mix and match different gas canister pressures, different medication depots and interchangeable tips means that a single kit can be provided with interchangeable and assemblable elements.

The TQD3 device according to this invention is susceptible to use for nasal and per rectum delivery routes for a range of medications, and is flexible enough to accommodate delivery requirements, fluid viscosity and total volume to be delivered. Applicators requiring high pressure to discharge properly may require a screw-on mechanism while for low-pressure applicators, snap-together or screw-on assembly mechanisms are acceptable.

It will be appreciated by those skilled in the art that the device, method and system according to this invention may be used in human or non-human subjects. Preferably, in all cases, the subject is a living subject as defined by a beating heart (it being less relevant for purposes of operation of the invention whether the subject is showing brain activity or not). Unlike inhalational delivery devices, which require adequate respiratory function, the TQD3 system requires only cardiovascular circulation, whether intrinsic or artificial, such as is provided by external heart-lung bypass.

It will further be appreciated by those skilled in the art that a wide variety of medications may be administered to mucosal surfaces of a subject, provided the medication can be provided in a liquid form (for, e.g. nasal and rectal administration) or a gel (rectal administration), and provided there is evidence of suitable absorption and efficacy without undue toxicity when delivered by this route of administration.

Medications for delivery using the method, device and system or kit according to this invention include, but are not limited to: insulin, naloxone, opiates, cialis, levitra, triptan, imitrex, adrenaline, atropine, flumazenil, any antidepressant when in ICU, isoprenaline, dexamathesone, dopamine, propranolol, digoxin, lidocaine, nifedipine, protamine, phenytoin, nikenamide, nirtroglycerine, lorazepam, clobezam, and other compounds in the classes represented by these exemplary medications.

It may further be beneficial to utilize a nasal or rectal delivery method according to the present invention with respect to patients who suffer from short bowel syndrome or “dumping syndrome” who may also be afflicted with ulcerative colitis or Crohn's disease, or any other abdominal intestinal condition resulting in shortening of the intestine, such as in carcinoma of the bowel, who often do not absorb drugs properly due to the rapid transit time in the bowel.

It will also be appreciated by those skilled in the art that, while the foregoing disclosure relates primarily to single use, disposable embodiments of the invention, with relatively minor modifications, multi-use embodiments of the device may be used to advantage. For example, where the housing, instead of a molded plastic, is rather a surgical steel or titanium housing, the device may be sterilized. In addition, with inclusion of a seal, a portion of the housing may be opened and closed to thereby permit access to and replacement of the gas cylinder with a new, charged and sealed gas cylinder. For quarter turn triggered embodiments, by unscrewing the upper mating component, the internal components may be accessed. Furthermore, those skilled in the art will appreciate that a different motive force for driving the medication may be employed, such as, for example, where a pump or the like is included in the housing. Such embodiments, however, are likely to add cost and complexity to the device, but may have utility where a multi-use embodiment of the device is preferred. For multi-use embodiments, the medication storage depot may be a replaceable cylinder or capsule, or the capsule or cylinder may be a readily refillable/rechargeable container.

Those skilled in the art will also appreciate that while this disclosure has substantially focused on delivery of medication to nasal and rectal mucosal surfaces, with minor modifications, the device, system and method of this invention may be adapted for delivery to other mucosal surfaces, such as sublingual.

Having generally and specifically described various embodiments of the invention herein above, those skilled in the art will appreciate that variations, modifications, permutations and combinations of this invention as disclosed and suggested herein come within the scope of this invention and the appended claims.

As disclosed herein, this invention provides, in one embodiment or another, or combinations thereof:

1. A single-use, disposable device for administration of drugs to the nasal mucosa
2. A device for delivering medication that does not require a needle, and is therefore readily disposable in a non-biohazardous fashion.
3. A device intended to deliver a consistent and standardized dose of the drug to the nasal mucosa.
4. A device where a hydraulic plunger delivers a precise and accurate amount of drug.
5. A device whose consistency of delivery is assured by design.
6. A device where the dose of drug administered is not controlled by the person using the device, rendering the drug non-abusable through repeated use.
7. A device where the dose of drug administered is not controlled by the person using the device; a consistent dose is emitted upon actuation of the device by means of a triggered mechanism such as a spring-loaded button or quarter-turn rotation of an external sleeve activating a pressured canister.
8. A device where administration of the drug does not require inhalation or any coordinated activity by the recipient.
9. A device that needs minimal training for its use.
10. A device that can be used in an emergency situation to administer drugs where administration by the parenteral (intra-venous, intra-muscular, sub-cutaneous, intra-arterial) route is not convenient or possible, due to exigent circumstances, time constraints, unavailability of patent blood vessels (due to the patient being elderly, pediatric or hypovolemic as limited examples), or due to limited access due to other equipment already attached to the patient, and the like.
11. A device that can be used in an emergency situation to administer drugs where administration by the parenteral (intra-venous, intra-muscular, sub-cutaneous, intra-arterial) is not possible because of a lack of access to intravenous sites.
12. A device ideal for emergency situations to administer drugs where administration by the parenteral (intra-venous, intra-muscular, sub-cutaneous, intra-arterial) is not possible because the person using the device is not suitably qualified (e.g. a parent, teacher, relative, caregiver, first-aider).
13. A device that can be used in an emergency situation. For example, adrenaline for acute anaphylaxis, naloxone to reverse opioid over-dose, anti-histamine drugs for severe acute allergic reactions, atropine for protection from chemical warfare agents.
14. A device that can be used in an emergency situation where the patient is unconscious or unable to self-administer drugs.
15. A device that can be used to administer medication to patients undergoing a tonic-clonic seizure, where intravenous access is difficult.
16. A device that can be stored for possible emergency use by trained or untrained personnel, for example but not limited to in schools, ambulances, police cars, workplace emergency first aid kits, military first aid kits, airplane emergency kits and the like.
17. The device is for single use in order to guarantee hygiene and to avoid cross-infection, as well as limits its abuse potential (for example, opioids for migraine relief).
18. TQD₃ is a rapid, uniquely useful route of administration of non-intravenous drugs
19. TQD₃ is uniquely administrable and currently not available as a system in human or veterinary medicine.
20. A device that can administer a drug when intravenous medication is not available, e.g. because it could not be stored properly or because the medication is not manufactured in a form adapted by other than the oral route.
21. A device that can be used in a non-hospital environment, e.g., base camp, war zone, school, public transport, airplanes.
22. A device that can dispense a wide variety of medications that have been pre-dosed.
23. A device adapted to deliver varying drug categories and doses, distinguishable by color coded cylinders, embossed labelling, digital identification codes and the like.
24. Provides very high C-max (maximum blood levels) and with a very short T-max (rapidly).
25. Provides pharmacologic uptake that is comparable to pulmonary inhalation and at least equivalent to, if not superior to, intravenous administration with respect to ease and speed of administration and access to the brain.
26. Provides nebulization system sufficiently powerful to effect nasal mucosa bilaterally even when inserted in one nostril.
27. Provides a precise fixed rate and quantity of delivery between all units.
28. Provides a design that eliminates need to establish iv access for drugs where iv formulation is available (i.e., convulsions)
29. Is operated by a flip top cap which can be operated in a single-handed fashion.
30. Is a precise quantal delivery system that nebulizes the patient's drug requirement via a venturi system.
31. May be configured in a single dose delivery system which is non-reusable, thereby making accidental overdosing impossible.
32. Has venturi ducts of varying sizes which allows for adjustable micronization from 20-200 microns droplet size.
33. Provides a delivery pressure designed to prevent loss of drug through unoccupied nostril.
34. Has an adjustable skirt on nasal cannula which provides a complete seal to allow for full delivery of drug in spite of nasal aperture.
35. The drug is fully administered with each use, preventing atmospheric contamination or bystander second-hand administration.
36. Is a self-contained delivery unit which requires one-step assembly prior to usage, making it valuable in time-sensitive or in high-stress situations.
37. Is operated by a push button activator which punctures a compressed air cylinder.
38. Has depressurization vents that allow the device to equalize at atmospheric pressure for safe disposal after use.
39. Has a pneumatic activator restraining cap which prevents early discharge/release of contained drug.
40. Contains double O ring seals which ensure predictable, controlled pressure in the device prior to the use of depressurizing vents that then vents after dispensing the medication.
41. Has a positioning spring which guides cartridges into a lance/pin that punctures compressed air cylinder for equal dispersion of gas. This spring ensures repulsion of the gas cylinder to ensure full discharge.
42. Has varying diameters of medicine storage cylinders to suit dosing requirements while maintaining hydraulic piston travel distance between units, or, alternatively, where varying piston lengths and travel distances accommodates different volumes of medication in the medication storage depot which may have consistent bore diameter as between different embodiments of the device.
43. Has a predictable, designed failure of a rupture-able disk, containing the medication in the device, which allows for consistent dosage dispersed from unit to unit.
44. Requires no special disposal, sanitary or otherwise.
45. Requires release of push button after depression to pressurize pneumatic activator.
46. When activated, the air pressure is sufficient within the device, to cause the pneumatic actuator O-ring to slide over the restraining catch, allowing travel of the piston to disperse the medication.
47. The device contains a hydraulic piston driven through the medicine storage cylinder by air pressure to discharge the medicine into patient's nostrils.
48. The rupture-able disk from the medicine cylinder which is designed to fail predictably, will not plug the nebulizing nozzle.
49. Depressurizing vents at sides of device prevent undesirable and unnecessary pressure build up behind the pneumatic plunger, once activated.
50. Once the pneumatic actuator O-ring passes the vents, the entire sprayer housing will completely depressurize device automatically, allowing for safe disposal.
51. Delivery technology is adjustable to any cartridge of drug in any dosage.
52. A device which requires ¼ turn of rotation cap clockwise to drive compressed air cylinder into lance and then a quarter turn counter clockwise to release the pneumatic pressure.
53. A single-use, disposable device for administration of drugs to the nasal mucosa.
54. A device where the dose of drug administered is not controlled by the person using the device; a consistent dose is emitted upon actuation of the device by means of a triggered mechanism such as a spring-loaded button or quarter-turn rotation of an external sleeve activating a pressured canister.
55. A device where administration of the drug does not require inhalation or any coordinated activity by the recipient.
56. Precise sterile drug delivery system by way of rectum.
57. Device designed to use an orifice (e.g. rectum) not otherwise occupied in clinically ill patients, e.g., ICU patients with no nasal access due to other medical hardware, e.g., oxygen mask or nasogastric tubes.
58. Device designed to deliver drugs where no i.v. formulation is available, e.g., many anticonvulsants, antidepressants (from which withdrawal can occur in an ICU setting or post operatively).
59. Protective, easily removable, sterile cover with pre-loaded lubricant allows for ease of insertion.
60. Allows for rapid application with single handed operation for per rectum delivery.
61. Allows for precise delivery of medication via rectum due to guided pneumatic actuator.
62. Accounts for anatomical variation in length of anal canal
63. Allows for clip or screw on medicine cartridges of any medicine or dose that can be stored in a system-appropriate form (e.g. liquid).
64. Pneumatic dose piston system has sufficient charge to ensure delivery of quantal dose to rectum.
65. Precise dose delivery system allows for full quantal delivery dose independent of operator strength or knowledge.
66. Clip on cylinder is a two-piece system which contains drug type and dosing that is color coded and imprinted on medicine cylinder.
67. 2-piece (pneumatic cartridge and medicine cylinder) assembly limits administration to single dose of drug.
68. 2-piece (pneumatic cartridge and medicine cylinder) assembly eliminates possibility of undesired drug combinations.
69. Requires applicator with medicine to be screwed or clipped on to delivery device once protective coverings are removed.
70. Removal of lubricant filled protective covering from applicator is now achieved via a simple motion.
71. Activation can only be achieved by ¼ turn clockwise, followed by ¼ turn counter-clockwise; therefore, not accidentally. Discharge vents prevent any gas from being discharged into rectum.
72. A device which allows drug access to animals by veterinarians or others by the rectal route of administration.
73. Is advantageous in this regard as it involves shaving an animal's hair down to skin to try and find venous access.
74. A device which circumvents the need for difficult oral administration of animal medications.
75. A device which would provide precise drug dose administration in the veterinarian setting.
76. The device whose caliber of rectal cannula could be adjusted widely to account for inter-species variation in anal canal diameter and length.
77. A device which allows the administration of medications essential to animals that have been anaesthetised and cannot take anything by mouth.
78. A device which would provide an optimal route of delivery to incapacitated animals or otherwise unable to receive oral medications.

Accordingly, the invention defined herein includes a device, a method, a system and a kit for delivery of medication to mucosal surfaces. The medication delivery device is adapted for delivery of a precise quantity of medication to mucosal tissue of a subject and includes:

a. a gas-tight housing comprising a proximal and a distal end, said distal end comprising a conduit through which medication is discharged;
b. a trigger mechanism incorporated into the proximal end of said housing wherein on actuation, a pressurized gas canister is perforated to release pressurized gas within said gas-tight housing;
c. a pneumatic actuator comprising a proximal end which forms a gas-tight seal within said gas-tight housing, and a distal end terminating in a shaft of piston, which, on exposure to said pressurized gas within said gas-tight housing, is induced to travel toward the distal end of said gas-tight housing;
d. a piston, either unitary with or juxtaposed to the distal end of said pneumatic actuator and which is free to move toward the distal end of said housing through a bore, upon being impelled to move by said pneumatic actuator;
d. in the case of the component system, a separate, attachable, sealed medication storage depot comprising a precise quantity of a medication, wherein said storage depot is defined at its proximal aspect by said piston, on either side by the walls of said storage depot within a bore through which said piston travels on being impelled by said pneumatic actuator, and by a rupture-able disc at its distal aspect;
e. a conduit adapted for mucosal delivery of medication through which medication is discharged upon said rupture-able disc being ruptured due to pressure from said piston driving said medication through said medication storage depot into said conduit for discharge at the distal end of said housing.

The medication delivery device may be a unitary device or it may be assembled from component parts. For example, the device may comprise two components, the first of which includes elements (a)-(c) and the second of which includes elements (d)-(e) as described above. The device may be adapted for nasal delivery of medication or for rectal administration of medication. For nasal delivery of medication, preferably, the device further comprises at least one pneumatic actuator restraining catch to define the exposure to pressurized gas within the gas-tight housing which is sufficient to induce the pneumatic actuator to travel toward the distal end of the gas-tight housing. The device is further adapted for nasal delivery of medication by including at the distal tip of the housing an atomizing nozzle for production of droplets of medication between about 20 microns to about 200 microns in diameter. In a preferred embodiment, the atomizing nozzle includes at least one and preferably at least two venturi ducts for drawing air into the stream of medication as it is discharged to assist in production of the droplets of medication. Further, a skirt applied to the distal end of the conduit is preferred to produce a gas-tight seal at the nostril of a patient when the conduit is inserted for medication delivery to the nasal mucosa.

Alternatively, the device is adapted for rectal delivery of medication by including a conduit covered by gel under an easily removable protective covering, preserving sterility to assist in insertion into the rectum of a patient.

For either route of delivery, in preferred embodiments, the trigger mechanism incorporated into the proximal end of the housing comprises (a) a protected pushbutton actuator which, on being un-protected and depressed, drives the pressurized gas canister into a lance which perforates the gas canister; or (b) a threaded cap which on being rotated approximately a quarter turn drives the pressurized gas canister into a lance which perforates the gas canister. When the device includes a protected pushbutton actuator, it is preferred that the device include a biasing spring such that upon release of the pushbutton actuator, the spring biases the now perforated gas canister away from the lance, to thereby release pressurized gas within the gas-tight housing. When the device includes a threaded cap trigger mechanism, the cap is rotated approximately a quarter turn back toward its original position to release pressurized gas within the gas-tight housing. In either embodiment of trigger mechanism included in the device, in a preferred embodiment, the lance is supported on a perforated support such that upon release of the pressurized gas within the gas-tight housing, pressure is applied to the pneumatic actuator to drive it forward, causing the piston to drive medication out of the distal conduit of the device and onto the mucosal surfaces.

When the device is provided in two components, these are assembled into a unitary and operative device via male-female connectors which are, preferably, sealingly engaged with each other by screwing together the parts bearing matching screw threads, snapping together interlocking parts or via a Luer lock mechanism.

The method according to the invention for delivering medication to a mucosal surface involves using a medication delivery device adapted for delivery of a precise quantity of medication to mucosal tissue of a subject, embodiments of which device are described above. For nasal delivery of medication, this involves

a. selecting and applying to the distal, conduit end of said housing an appropriately sized sealing skirt to achieve a gas-tight seal when said conduit is inserted into the nostril of a patient;
b. inserting the conduit into the nostril of a patient; and
c. actuating said trigger mechanism to deliver said medication into the nostril of the patient.

For rectal delivery of medication, this involves:

(a) removing any protective cover included to protect said conduit and gel covering said conduit;
(b) inserting said conduit into the rectum of a patient; and
(c) actuating said trigger mechanism to deliver said medication into the rectum of the patient.

The invention also includes a system for delivering a precise quantity of medication to mucosal tissue of a subject which includes the embodiments of the device described herein above, while a kit according to the invention includes embodiments of the device described herein above, including components thereof which are assembled prior to use, which facilitates a mix-and match approach as appropriate for delivery of a desired dose of medication via a route selected from nasal or rectal delivery.

Considerable flexibility is provided by different embodiments according to this invention, according to which application nozzles adapted for different routes of mucosal administration are interchangeable, as are the doses and composition of medications.

In an exemplary and non-limiting application of an embodiment of the present invention, an embodiment adapted for intra-nasal administration of naloxone is optimally presented to the olfactory mucosa, located in the upper nasal cavity, (just below the cribriform plate of the skull, containing olfactory cells which traverse the cribriform plate and extend up into the cranial cavity). Atomized/nebulized by the device according to this invention, there is provided a method and system for delivering a mist comprising micro-droplets (about 20 to 200 micron) containing medication molecules. The device deploys these droplets to contact this specialized mucosa whereby they are rapidly transported directly into the brain, skipping the blood-brain barrier, and achieving very rapid cerebrospinal fluid levels (often faster than if the drug is given intravenously). Accordingly, this invention efficiently enables nose-brain pathway delivery of medication in any situation in which rapid and safe delivery of centrally acting medications is called for. This class of medications and the situations implied by their use include but are not limited to, sedatives, anti-seizure drugs, opiates, particular brain receptor agonists or antagonists, and the like are, delivered intra-nasally according to this invention.

Multiple authors demonstrate that the nose-brain pathway leads to nearly immediate delivery of some nasal medications to the cerebral spinal fluid, by-passing the blood brain barrier, and all of these applications are amenable for use of an embodiment of the present invention. See, e.g. Henry, R. J., et al., A pharmacokinetic study of midazolam in dogs: nasal drop vs. atomizer administration. Pediatr Dent, 1998. 20(5): p. 321-6; Sakane, T., et al., Transport of cephalexin to the cerebrospinal fluid directly from the nasal cavity. J Pharm Pharmacol, 1991. 43(6): p. 449-51; Banks, W. A., M. J. During, and M. L. Niehoff, Brain uptake of the glucagon-like peptide-1 antagonist exendin(9-39) after intranasal administration. J Pharmacol Exp Ther, 2004. 309(2): p. 469-75; Westin, et al., Direct nose-to-brain transfer of morphine after nasal administration to rats. Pharm Res, 2006. 23(3): p. 565-72). Alternate embodiments according to this invention are adapted for applications in the intra-rectal delivery for quick, safe and effective systemic dosing via the highly vascularized rectal mucosa.

In summary, the medication delivery device, system and method according to this invention is directed to delivery of medication via the underutilized mucosal routes of delivery, including but not limited to intra-nasal, per rectum, and in appropriately adapted variations, other mucosal surfaces such as the sublingual surface. In certain embodiments, the delivery system according to this invention comprises interchangeable cartridges that are suitable to access these underutilized highly vascularized areas. The system is easy to use and safe from a biohazard production perspective, given that it is a needle-free drug delivery device. In particular embodiments, drug cartridges that are, e.g. color-coded, and/or embossed clearly with information about the contents of the cartridge. Single use and multiple-use, rechargeable embodiments of the invention are contemplated as coming within the scope of this invention disclosure and appended claims.

What is claimed is:

1. A medication delivery device adapted for delivery of a precise quantity of medication to mucosal tissue of a subject comprising:
a. a gas-tight housing comprising a proximal and a distal end, said distal end comprising a conduit through which medication is discharged;
b. a trigger mechanism incorporated into the proximal end of said housing wherein on actuation, a pressurized gas canister releases pressurized gas within said gas-tight housing;
c. a pneumatic actuator comprising a proximal end which forms a gas-tight seal within said gas-tight housing, and a distal end terminating in a shaft leading to a piston, which, on exposure to said pressurized gas within said gas-tight housing, is induced to travel toward the distal end of said gas-tight housing;
d. a piston, either unitary with or juxtaposed to the distal end of said pneumatic actuator and which is free to move toward the distal end of said housing through a bore, upon being impelled to move by said pneumatic actuator;
e. a sealed medication storage depot comprising a precise quantity of a medication, wherein said storage depot is defined at its proximal aspect by said piston, on either side by the walls of said storage depot within a bore through which said piston travels on being impelled by said pneumatic actuator, and by a rupture-able disc at its distal aspect;
f. a conduit adapted for mucosal delivery of medication through which medication is discharged upon said rupture-able disc being ruptured due to pressure from said piston driving said medication through said medication storage depot into said conduit for discharge at the distal end of said housing.

2. The medication delivery device according to claim 1 wherein said device is unitary or is assembled from component parts.

3. The medication delivery device according to claim 2 wherein said device comprises two components, the first of which includes elements (a)-(c) and the second of which includes elements (d)-(f) of claim 1.

4. The medication delivery device according to claim 1 adapted for nasal delivery of medication or for rectal administration of medication.

5. The medication delivery device according to claim 4 adapted for nasal delivery of medication, further comprising at least one pneumatic actuator restraining catch to define the exposure to said pressurized gas within said gas-tight housing which is sufficient to induce said pneumatic actuator to travel toward the distal end of said gas-tight housing.

6. The medication delivery device according to claim 5 further adapted for nasal delivery of medication, comprising, at the distal tip of said housing, an atomizing nozzle for production of droplets of medication between about 20 microns to about 200 microns in diameter.

7. The medication delivery device according to claim 6 wherein said atomizing nozzle comprises at least one venturi duct for drawing air into the stream of medication as it is discharged to assist in production of said droplets of medication.

8. The medication delivery device according to claim 1 comprising a skirt applied to the distal end of said conduit to produce a gas-tight seal at the nostril of a patient when said conduit is inserted therein for medication delivery thereto.

9. The medication delivery device according to claim 4 adapted for rectal delivery of medication, further comprising a conduit covered by a gel to assist in insertion into the rectum of a patient.

10. The medication delivery device according to claim 1 wherein said trigger mechanism incorporated into the proximal end of said housing comprises (a) a protected pushbutton actuator which, on being un-protected and depressed, drives said pressurized gas canister into a lance which perforates said gas ganister; or (b) a threaded cap which on being rotated approximately a quarter turn drives said pressurized gas canister into a lance which perforates said gas canister.

11. The medication delivery device according to claim 10 wherein in (a) said protected pushbutton actuator further comprises a biasing spring such that upon release of said pushbutton actuator, said spring biases the now perforated gas canister away from said lance, to thereby release pressurized gas within said gas-tight housing; or (b) said threaded cap, on being rotated approximately a quarter turn back toward its original position, thereby releases pressurized gas within said gas-tight housing.

12. The medication delivery device according to claim 11 wherein said lance is supported on a perforated support such that upon release of said pressurized gas within said gas-tight housing, pressure is applied to said pneumatic actuator.

13. The medication delivery device according to claim 3 wherein said two components are assembled into a unitary and operative device via male-female connectors.

14. The medication delivery device according to claim 13 wherein said male-female connectors are sealingly engaged with each other by a mechanism selected from the group consisting of matching screw threads, an interlocking snap mechanism, and a Luer lock mechanism.

15. A method for delivering medication to a mucosal surface which comprises using a medication delivery device adapted for delivery of a precise quantity of medication to mucosal tissue of a subject comprising:
a. a gas-tight housing comprising a proximal and a distal end, said distal end comprising a conduit through which medication is discharged;
b. a trigger mechanism incorporated into the proximal end of said housing wherein on actuation, a pressurized gas canister releases pressurized gas within said gas-tight housing;
c. a pneumatic actuator comprising a proximal end which forms a gas-tight seal within said gas-tight housing, and a distal end terminating in a shaft of piston, which, on exposure to said pressurized gas within said gas-tight housing, is induced to travel toward the distal end of said gas-tight housing;
d. a piston, either unitary with or juxtaposed to the distal end of said pneumatic actuator and which is free to move toward the distal end of said housing through a bore, upon being impelled to move by said pneumatic actuator;
e. a sealed medication storage depot comprising a precise quantity of a medication, wherein said storage depot is defined at its proximal aspect by said piston, on either side by the walls of said storage depot within a bore through which said piston travels on being impelled by said pneumatic actuator, and by a rupture-able disc at its distal aspect;
f. a conduit adapted for mucosal delivery of medication through which medication is discharged upon said rupture-able disc being ruptured due to pressure from said piston driving said medication through said medication storage depot into said conduit for discharge at the distal end of said housing.

16. The method according to claim 15 which comprises, for nasal delivery of medication:
a. selecting and applying to the distal, conduit end of said housing an appropriately sized sealing skirt to achieve a gas-tight seal when said conduit is inserted into the nostril of a patient;
b. inserting the conduit into the nostril of a patient; and
c. actuating said trigger mechanism to deliver said medication into the nostril of the patient.

17. The method according to claim 15 which comprises, for rectal delivery of medication:
(a) removing any protective cover included to protect said conduit and gel covering said conduit;
(b) inserting said conduit into the rectum of a patient; and
(c) actuating said trigger mechanism to deliver said medication into the rectum of the patient.

18. A system for delivering a precise quantity of medication to mucosal tissue of a subject comprising:
a. a gas-tight housing comprising a proximal and a distal end, said distal end comprising a conduit through which medication is discharged;
b. a trigger mechanism incorporated into the proximal end of said housing wherein on actuation, a pressurized gas canister releases pressurized gas within said gas-tight housing;
c. a pneumatic actuator comprising a proximal end which forms a gas-tight seal within said gas-tight housing, and a distal end terminating in a shaft of piston, which, on exposure to said pressurized gas within said gas-tight housing, is induced to travel toward the distal end of said gas-tight housing;
d. a piston, either unitary with or juxtaposed to the distal end of said pneumatic actuator and which is free to move toward the distal end of said housing through a bore, upon being impelled to move by said pneumatic actuator;
e. a sealed medication storage depot comprising a precise quantity of a medication, wherein said storage depot is defined at its proximal aspect by said piston, on either side by the walls of said storage depot within a bore through which said piston travels on being impelled by said pneumatic actuator, and by a rupture-able disc at its distal aspect;
f. a conduit adapted for mucosal delivery of medication through which medication is discharged upon said rupture-able disc being ruptured due to pressure from said piston driving said medication through said medication storage depot into said conduit for discharge at the distal end of said housing.

19. A kit for delivery of a precise quantity of medication to mucosal tissue of a subject comprising:
(a) the device according to claim 1; and
(b) component parts thereof for assembly in a mix-and match fashion as appropriate for delivery of a desired dose of medication via a route selected from nasal or rectal delivery.
